(19) United States
(12) Patent Application Publication
(10) Pub. No.: US 2009/0209828 A1
(43) Pub. Date: Aug. 20, 2009

(54) METHOD AND DEVICE MICROCALORIMETRICALLY MEASURING A TISSUE LOCAL METABOLISM SPEED, INTRACELLULAR TISSUE WATER CONTENT, BLOOD BIOCHEMICAL COMPONENT CONCENTRATION AND A CARDIO-VASCULAR SYSTEM TENSION

(76) Inventor: Ramil Faritovich Musin, Moscow (RU)

Correspondence Address: FISH & RICHARDSON PC, P.O. BOX 1022, MINNEAPOLIS, MN, 55440-1022, US

(21) Appl. No.: 11/908,073

(22) PCT Filed: Mar. 9, 2005
(86) PCT No.: PCT/RU05/00039

§ 371 Date: Mar. 20, 2009

(51) Int. Cl. A61B 5/01; A61B 5/00

(52) U.S. Cl. 600/301; 600/307

(57) ABSTRACT

The present invention relates to medicine, in particular, to the methods of measuring a thermal effect and a local metabolism rate of a live tissue, a water content in the intercellular substance as well as a concentration of biochemical blood components, in particular, blood glucose and pressure in the cardiovascular system.

The present invention relates to medicine, in particular to methods for the measurement of thermal effect and local metabolism rate of live tissue, intercellular substance water content, blood concentration of biochemical ingredients, in particular blood glucose level and pressure in the cardiovascular system.

### DESCRIPTION OF PRIOR ART

According to the American Diabetic Association, about 6% of the US population, i.e. about 16 million persons suffer from diabetes mellitus. According to the reports of the same Association, diabetes is the sevenths main diseases resulting in lethal outcome in the USA. The number of deaths caused by diabetes is about 200,000 annually. Diabetes is a chronic disease the method of treating which are currently still at the development stage. Diabetes often leads to the development of complications such as blindness, renal disorders, nervous diseases and cardiovascular diseases. Diabetes is a leading disease resulting in blindness at the age of 20 to 74 years. Ap[proximately from 12,000 to 24,000 persons annually loss vision because of diabetes. Diabetes is a leading cause of renal diseases in about 40% of new cases. About 40 to 60% of patients with diabetes are predisposed to different forms of nervous diseases, which can result in amputation of limbs. Patients with diabetes are approximately 2 to 4 fold more predisposed to cardiac diseases, in particular myocardial infarction.

Diabetes is a disease associated with insufficient production or inefficient use of insulin by cells of the body. In spite of the fact that causes of the disease are not completely understood, some factors such as genetic, environmental, viral have been identified.

There are two main forms of diabetes: type 1 and type 2.

Type 1 diabetes (known as insulin-dependent diabetes) is an autoimmune disease wherein insulin production completely terminates; and it most often develops in childhood and youth. Patients with type 1 diabetes need daily insulin injections.

Type 2 diabetes is a metabolic disease caused by that the body cannot produce a sufficient amount of insulin or utilization thereof is inefficient. Patients with type 2 diabetes make up about 90 to 95% of a total amount of diabetics. Morbidity of type 2 diabetes in the USA approaches an epidemiologic threshold, mainly due to increase in the number of elderly Americans and a significant prevalence of a hypodynamic life style and obesity.

Insulin promotes glucose penetration into a cell with subsequent cleavage thereof to obtain energy for all metabolic processes. In diabetics, glucose cannot penetrate into a cell, it accumulates in blood and cells experience energetic hunger.

Patients with type 1 diabetes inject to themselves insulin using a special syringe and a cartridge. Continuous subcutaneous injection of insulin through an implanted pump is also possible. Insulin is typically prepared from swine pancreas or it is synthesized chemically.

Attending physicians insistently advise that patients taking insulin should provide self-monitoring blood sugar level. Knowing blood sugar level, patients can adjust insulin dose in subsequent injection. Adjustment is necessary, since because of different reasons, blood sugar level fluctuates during a day and from day to day. In spite of the importance of such monitoring, several conducted studies showed that a portion of patients who carry out such monitoring at least once daily, diminishes with age. This fall occurs mainly because the currently used method of monitoring is associated with invasive drawing a blood sample from a finger. Many patients consider drawing a blood sample from a finger to be a more painful procedure that insulin injection.

The methods and devices for measuring blood sugar level are known: [19-24].

The proposed method and device for embodiment thereof allow determining blood sugar level by measurement using a calorimetric method of thermal effect (heat production) and a local tissue metabolism rate. Existence of the functional relationship between sugar absorption rate by tissue cells and blood level thereof is indicated in the works [2,8,9].

The following methods: direct calorimetry and indirect calorimetry are the known methods of physiological calorimetry [16].

The method of direct calorimetry contemplates immediate determination of a total amount of irradiated heat using a calorimetric chamber for live objects.

The method of indirect calorimetry allows for determining an amount of irradiated heat in an indirect way based on accounting respiratory gas exchange dynamics using respiratory chambers and different systems. Two possible modifications of the indirect calorimetry method are distinguished: a method of a complete gas analysis (accounting absorbed O₂ and evolved CO₂) and a method of incomplete gas analysis (accounting absorbed O₂).

The closest to the claimed object by chemical essence and achievable result is the method of the basal metabolic rate of the human body using a whole body calorimeter (a direct calorimetry) described in [26]. (Determination of the basal metabolic rate of humans with a whole body calorimeter. U.S. Pat. No. 4,386,604). By change in air temperature and a total water amount evaporating from the whole body surface, a total whole body heat irradiation is determined and the basal metabolic rate is calculated.

Another closest to the claimed object by chemical essence and achievable result is the method of measurement described in [25] (Whole body calorimeter., U.S. Pat. No. 5,040,541).

The main drawbacks of the mentioned methods consist in that for embodiment thereof, cumbersome, stationary and expensive whole body calorimetric chambers are required. Furthermore, the direct calorimetry method is characterized by a low accuracy.

The present invention is aimed at enhancement of measurement accuracy.

The set object is achieved by that thermal effect of local tissue metabolism is measured and blood sugar level is determined. A value of thermal effect is determined by measuring a total amount of water evaporated from the skin surface during non-perceived perspiration and by measuring an ambient temperature.

### LIST OF DRAWINGS

FIG. 1 shows a diagram of relationship between osmotic pressure of intercellular substance and hydraulic capillary pressure and the non-dimensional parameter α=P₀/P.

FIG. 2 shows a diagram of relationship between elastic strain of intercellular substance (elastic pressure) and intracapillary hydraulic pressure.

FIG. 3 shows a diagram of relationship between osmotic pressure of intercellular substance and hydraulic capillary pressure and a non-dimensional parameter “α” for different values of blood glucose concentration.

FIG. 4 shows relationship between elastic strain of the intercellular substance (elastic pressure) and a non-dimensional parameter “α” for different values of blood glucose concentration.

FIG. 5 shows a diagram of relationship between intracapillary hydraulic pressure and blood glucose concentration. By the ordinate axis, hydraulic capillary pressure in mm Hg relative to atmospheric pressure is plotted. By the abscissa axis, blood sugar value in mM per 1 liter is plotted.

FIG. 6 shows an equivalent scheme of a device for measuring water amount in the intercellular substance using the electrometric method.

FIG. 7 shows a photograph of a general view of the experimental instrument for non-invasive measurement of blood sugar level and local tissue metabolism rate.

FIG. 8 shows a characteristic time course of transverse electric conductivity of the epidermal corneous layer (ECL) caused by swelling process of the intercellular substance.

FIG. 9 shows correlation between indications of the experimental instrument with indications of a standard glucometer by the results of 15 experiments carried out on one practically healthy tested person. The glucometer “Accu Chek Active” was used for control measurements. A total number of control measurements by blood samples in 15 experiments was 38 measurements. All measurements were done using one calibration. Indications of the experimental instrument in the time points corresponding to the time points of control measurement by blood samples drawn from a finger, coincide with indications of the certified glucometer with accuracy of 1-2% that was caused by index error of the latter. Typical results of such experiments carried at different time during a day as well as at different days are presented in FIGS. 10-14.

FIG. 10 shows typical results of comparative measurements: time course of blood sugar level performed using the experimental instrument in the monitoring regimen (the red curve, frequency of measurements 6 seconds) and the standard glucometer “Accu Chek Active” manufactured by the firm Roche Diagnosis GmbH (grey rectangles). Accuracy of the glucometer “Accu Chek Active” measuring blood sugar level by photometric method (by blood samples drawn from a finger) is 1-2%. The diagrams present the results of two experiments on measurement of blood sugar level in a practically healthy patient during a day: the first curve (from 12:00 to 13.30) illustrates time course of blood sugar level in about 30-40 minutes following food consumption during dinner. A total number of measurements by blood samples in these experiments was 7 measurements (at the time point 13:20 during the first experiment three measurements from one sample were done).

FIG. 11 shows the glucose tolerance test results (“a sugar curve”) in a practically healthy patient (the first diagram in FIG. 10). The red curve demonstrates time course of blood sugar level recorded in the monitoring regimen using the experimental instrument; the results of measurements performed using the “Accu Chek Active” instrument are shown by grey rectangles. The time point of sugar loading is marked by the arrow.

FIG. 12 shows time course of blood sugar level in a practically healthy patient 30 minutes after dinner (the second diagram in FIG. 10).

FIG. 13 shows diagrams presenting the results of two experiments (prior to and post supper) of measuring blood sugar level in a practically healthy patient: the first curve (from 20:30 till 21:00)—changes in blood sugar level prior to supper; the second curve (from 22:00 till 22:30)—time course of blood sugar level approximately 20-30 minutes post supper.

FIG. 14 shows the glucose tolerance test results (“a sugar curve”) in a practically healthy patient. The time point of sugar loading is marked by the arrow.

FIG. 15 shows the correlation diagram between indications of the experimental instrument and indications of the control glucometer by the results of four experiments carried out on one patient D1 with type 1 diabetes (a 55 year old woman). The “Accu Chek Active” glucometer was used for control measurements. A total number of control measurements by blood samples in four experiments was 21. All the measurements were done using one calibration. Indications of the experimental instrument in the time points corresponding to the time points of control measurement by blood samples drawn from a finger coincide with indications of the certified glucometer with accuracy which is determined by index error of the latter (1-2%). Typical results of these experiments carried at different days are presented in FIGS. 16-17.

FIG. 16 shows time course of blood sugar level in the patient D1 1.5 hr post supper.

FIG. 17 shows time course of blood sugar level in the patient D1 1.5 hr prior to supper.

FIG. 18 shows the correlation diagram between indications of the experimental instrument and indications of the control glucometer by the results of four experiments carried out on one patient D2 with type 2 diabetes (a 76 year old man). The “Accu Chek Active” glucometer was used for control measurements. A total number of control measurements by blood samples in four experiments was 21. All the measurements were done using one calibration. Indications of the experimental instrument in the time points corresponding to the time points of control measurement by blood samples drawn from a finger coincide with indications of the certified glucometer with accuracy which is determined by index error of the latter (1-2%). Typical results of these experiments carried at different days are presented in FIGS. 19-20.

FIG. 19 shows time course of blood sugar level in the patient D2 immediately post supper.

FIG. 20 shows time course of blood sugar level in the patient D2 post dinner.

FIG. 21 shows typical time course of water content in the intercellular substance during muscular exercises.

FIG. 22 shows relationship between water content in the intercellular substance and external pressure.

FIG. 23 shows relationship between water content in the intercellular substance (and water flow density through ECL) and external heat flow.

FIG. 24 shows a typical time course of water content in the intercellular substance in local effect on a surface of heat flows. By the abscissa axis, time in seconds is indicated, by the ordinate axis water content in the epidermal corneous layer in relative units are indicated. Beginning (a) and termination (b) of the effect are marked with arrows. “1” indicates local heating using heat flow “+”10 mWt/cm²; 2 and 3 indicate local cooling using heat flow “−”10 mWt/cm².

FIG. 25 shows relationship between water content in the intercellular substance and blood sugar level.

FIG. 26 shows typical examples of the cardiovascular system disorders.

FIG. 27 shows a photograph of a general view of the instrument for local decompression.

FIG. 28 shows time course of water content in the intercellular substance during exposure of the body surface to local decompression. Local decompression causes constriction of the intercellular substance volume under the applicator.

FIG. 29 shows time course of tissue sugar absorption rate and heat production during glucose tolerance test. The red and blue diagrams are monitoring curves obtained using the experimental instrument, a two-channel micro calorimeter. The time point of oral sugar loading is marked with the arrow. The distance between measuring sensors is 12 cm. Originating from the analysis of the curves, one can see that temporal changes in heat production of two tissue sites disposed close to each other are practically synchronous. Temporal delay between the monitoring curves does not exceed 100 seconds.

FIG. 30 shows a drawing clarifying a registration method of two-dimensional spatial-temporal distribution of local metabolism rate using a multi-channel matrix of sensors (16 channels 4×4).

FIG. 31 shows two-dimensional spatial-temporal distribution of local metabolism rate obtained using the multi-channel matrix of sensors (16 channels 4×4). The presented results clarify the method of dynamic mapping local tissue metabolism rate.

FIG. 32 shows visualization of therapeutic effect using real-time multi-channel recording.

FIG. 33 shows visualization of therapeutic effect using the dynamic mapping method.

FIG. 34 shows spatial-temporal distribution of water content in the intercellular substance in gastric ulcer disease.

### DETAILED DISCLOSURE OF THE INVENTION

### Physical Basis of Live Tissue Heat Exchange with the Environment

Heat exchange is a spontaneous and irreversible process of heat transfer caused by temperature gradient. The following forms of heat exchange are distinguished: heat conductivity, convection, radiant heat exchange, heat exchange in phase conversions.

Heat transfer is heat exchange between the body surface and a medium (liquid, gas) contacting therewith.

Evaporative cooling is heat exchange between tissue and the environment caused by evaporation of water delivered to the epidermis from deep tissue layers. Heat flow density is determined by product of evaporation heat (steam generation heat) by water flow density evaporating from the surface.

Radiant heat exchange (radiation heat exchange, radiant transfer) is energy transfer from one body to another caused by the processes of emission, propagation, scattering and absorption of electromagnetic radiation. Each of these processes adhere to definite regularities.

Thus, under the conditions of equilibrium heat radiation emission and absorption adhere to the Plank's law of radiation, to the Stephan-Boltzman law, to the Kirgoff law of radiation.

Essential difference of radiant heat exchange form the other forms of heat exchange (convection, heat conductivity) consists in that it can occur in the absence of a material medium separating surfaces of heat exchange, as electromagnetic radiation also propagates under vacuum.

The Plank's law of radiation establishes relation between radiation intensity, spectral distribution and temperature of the black body. In elevation of temperature, radiation energy rises. Radiation energy depends on wavelength. A total energy irradiated by the black body and measurable by a contact-less infrared thermometer is a total energy irradiated at all wavelengths. It is proportional to the Plank's equation integral by wavelengths and it is described in physics by the Stephan-Boltzman's law.

The Stephan-Boltzman's radiation law asserts a fourth degree proportionality of the absolute temperature T of the full volume density ρ of the equilibrium radiation: ρ=α*T⁴, wherein “α” is a constant,

and a full emission capability W associated therewith:

W=β*T⁴, wherein “β” is the Stephan-Boltzman's constant.

Radiant heat exchange between tissue surface and the environment is determined by the ratio:

ΔW=β*(Tₜᵢₛₛᵤₑ⁴−Tₐᵢᵣ⁴)=W₀*(4ΔT/T)=W₀*[4(Tₜᵢₛₛᵤₑ−Tₐᵢᵣ)/Tₜᵢₛₛᵤₑ]

ΔT<<Tₜᵢₛₛᵤₑ

Tₜᵢₛₛᵤₑ is skin surface temperature,

Tₐᵢᵣ is ambient air temperature.

W₀=β*Tₜᵢₛₛᵤₑ⁴.

ΔW is heat radiation from tissue surface to the environment.

Heat conductivity is one of the forms of heat transfer from more heated body parts to less heated parts. Heat conductivity results in leveling temperature. In heat conductivity, energy transfer is effected as a result of direct energy transfer from particles having a greater energy to particles with a lower energy. If relative change in T at an average free run distance length of particles is small, then the main heat conductivity law (the Fourier's law) is fulfilled: heat flow density q is proportional to temperature gradient T: Q=−λ*grad T,

wherein λ is heat conductivity coefficient of heat conductivity independent on grad T. The λ coefficient depends on aggregate sate of a substance, molecular structure, temperature, pressure, composition thereof etc

Convection is heat transfer in liquids and gases by substance flows. Convection results in leveling substance temperature. In stationary heat delivery to the substance, stationary convection flows occur therein. Intensity of convection depends on difference of temperatures between layers, heat conductivity and viscosity of medium.

Evaporative cooling is heat exchange between tissue and the environment caused by evaporation of water delivered to the epidermis surface from deep tissue layers through water transport by intercellular space. Heat flow density is determined by the product of steam heat (steam generation heat) by flow density of water evaporating from the surface.

In a comfortable temperature zone under normal conditions, water transport by sweating is known to be practically absent and the main contribution into evaporative cooling process is determined by water transport to the body surface. In physiology and medicine, this process is known as non-perceivable perspiration [16].

Non-perceivable perspiration of water is observed under so called “comfortable conditions”:

| | | |
|---|---|---|
| Ambient air temperature: | 18-25° | C. |
| Atmospheric pressure: | 740-760 | mm Hg. |

Intensity of evaporative cooling process under comfortable conditions is known to make up 400 to 700 mL/day or 10⁻⁸ to 10⁻⁷ g/second*cm². This corresponds to values of heat flows 1 to 10 m Wt/cm².

Physical mechanisms of water transfer process to the body surface that provides maintaining heat balance of a local tissue site, are given consideration in the sections “Biophysical fundamentals: the mechanism of water transport through the epidermis” and “Biophysical fundamentals: the mechanism of a non-diffusion heat transfer from depth to surface”.

The Mechanism of Heat Transfer from Depth to Surface The results of experimental studies carried out by the inventors directly indicate to the mechanism of a non-diffusion transfer of heat generated during cellular metabolism, to the body surface. This mechanism has the following peculiarities:

1. A resulting transcapillary flow of water delivered from a capillary vessel into intercellular substance, is transferred by intercellular space to the body surface and maintains the process of evaporative cooling.

2. Heat generated during cellular metabolism is absorbed by flow of water circulating in intercellular space (due to a high heat capacity thereof), it is transferred from deep layers to the body surface and scattered into the environment during evaporation of water from the surface.

Physical mechanisms of heat transfer from deep layers to the surface are given detailed consideration in the section “Biophysical fundamentals: the mechanism of a non-diffusion heat transfer from depth to surface”.

The Mechanism of Maintaining Live Tissue Temperature

Constant maintaining heat content of live tissue is provided by the balance between generated heat (heat production) and heat irradiated into the environment (heat emission):

M+R+C+T+E=Q

wherein

M is heat production/,

R is heat emission by radiation (radiant heat exchange),

C is heat emission by convection,

T is heat emission by heat conductivity,

E is heat emission by evaporation (evaporative cooling),

Q is heat content.

Under the conditions of stationary equilibrium, heat content is equal to zero (Q=0) and tissue temperature is constant (T=const).

Resulting from the conducted experimental studies, main regularities determining relationship between water flow density through the epidermal corneous layer (ECL), ambient temperature and heat production of a live tissue:

In elevation of ambient temperature (at a constant level of heat production), reduction in heat emission occurs linear with it that is caused by difference of temperatures (radiation, heat conductivity and convection). Simultaneously, increase in heat emission occurs linear with rise in temperature due to evaporation in such way that a resulting heat balance and tissue temperature remain constant.

In rising heat production (at a constant ambient temperature), increase in evaporative cooling occurs linear with it in such way that a resulting heat balance and tissue temperature remain constant.

Physical mechanisms providing for maintaining heat balance of a local tissue site are given consideration in the sections “Biophysical fundamentals: physics of intercellular substance” and “Biophysical fundamentals: the mechanism of water transport through the epidermis”.

Physiologic and biochemical fundamentals of heat production in live tissue Oxidation of glucose which is one of the main energy suppliers in the body, occurs in accordance with the equation that may be presented in the following form:

Glucose+Oxygen=>CO₂+H₂O.

Change in a standard free energy in this reaction under physiologic conditions equals to:

ΔG=−686,000 cal/mole.

For comparison, a male weighing seventy kilograms who goes upstairs for an hour, expends about 1,000,000 cal. From this, it is clear that 686,000 cal. Mentioned above are a vast amount of energy. Work done by man is of course much less than energy expended during this work as in irreversible process, not all change in free energy is converted into work. Real efficacy of this conversion (as will be described below) is not higher than 40%. Moreover, food is not “burned” immediately in oxygen releasing energy in the form of heat and this release occurs in steps and includes a number of rather complex chemical conversions each of which gives a small “portion” of energy.

Glucose is oxidized in the body forming carbon dioxide and water; this is one of the most universal processes underlying respiration and digestion processes.

In breaking each glucose molecule accompanied by lowering free energy, energy is released that is sufficient to form 93 ATP molecules by binding of phosphate groups to ADP molecules. Not all 93 molecules appear to be actually formed. At the same time, all the process includes a large number of enzymatic reactions. Nutrients (carbohydrates, fatty acids and amino acids) enter into a series of reactions forming the Krebs cycle (or the cycle of tricarboxylic acids) during which carbonic backbone of molecules is broken down with formation of CO₂ but ATP is not formed here. On the following reaction steps transfer of electrons using special enzymes (respiratory chain) occurs. At these steps, ATP is synthesized and the last step on the way of a long process of electron transfer consists in binding thereof to molecular oxygen. Generally, electron transfer process along the respiratory chain resulting in accumulation of energy in ATP molecules is called oxidative phosphorylation. As a result of this process, 38 molecules of ATP as calculated per every consumed glucose molecule are formed. Efficacy of such transformation equals to 38/93=40%.

A value of heat production or heat power of the body can be quantitatively assessed originating from the following simple considerations.

An energetic value of human nutrition is about 2,400 kilocal. Daily. In a first approximation, 2,400 kilocal.=10⁴ J, 1 day (24 hrs)=86,400 seconds=1 seconds.

Then energy consumed by human body per one second will be 104/105=0.1 kJ*s⁻¹ or 100 J*s⁻¹, or 100 Wt; thus, heat power of a man is approximately equal to power of an electric bulb having power 100 Wt.

In muscular contraction, ATP which is energy donor for muscular contraction process, during reaction with myosin, allows for obtaining at most 50 J*g⁻¹ energy. This means that an ideal muscular system (i.e. with efficiency equal to 100%) for lifting a load weighing 1 kg to a 5 m height, would require expenditure of 2*10⁻³ mole ATP. Actually, muscular efficiency is about 30-40% and the rest portion is released in the form of heat.

Under normal conditions of the body's vital activity, glucose is a main energetic substrate. Normal human blood plasma glucose concentration depending upon nutrition conditions is maintained within the limits of 50 to 120 mg %. Postprandial glucose concentration in the portal vein system during absorption phase can achieve more than 270 m %. Elevation of blood glucose level always causes increase in insulin secretion.

In resting human body, fasting glucose metabolism rate averages 140 mg/hr per 1 kg body mass, 50% glucose being consumed by the brain, 20% by muscles, 20% by red blood cells and kidneys, and only 10% glucose are left for the rest tissues.

Glucose utilization rate (metabolism rate) in healthy man is a linear function of blood plasma glucose concentration. A mathematical relationship between glucose utilization and blood concentration thereof in normal humans is expressed by the equation:

Rᵤ=0.02554C+0.0785,

And in patients with non-ketotic diabetes:

Rᵤ=0.004448C+2.006,

wherein Rᵤ is glucose utilization rate in mg/min per 1 kg body mass, and C is blood plasma glucose concentration in mg % [Reichard G. A. et al., 1963; Forbath N., Hetenui C., 1966; Moorhouse J. A., 1973; Moorhouse J. A., et al., 1978; Hall S. E., et al., 1979, [2, 8, 9].

The term glucose “utilization” in physiological sense means the rate of glucose transport from blood into a general fund of tissue glucose and exit from it during metabolism. From biochemical point of view, glucose utilization rate is determined by transport through cytoplasmic membrane and by intracellular oxidative phosphorylation of glucose. The terms “turnover rate”, “assimilation” and “consumption” of glucose which are widely spread in the literature are synonyms of the notion glucose “utilization” and they are in any respect equivalent.

Under physiologic conditions, practically in all tissues glucose transport from intercellular medium into a cell is a first limiting reaction in glucose utilization by cells as in the absence of insulin, flow of transportable glucose is always less than glucose phosphorylation rate. Equilibrium between glucose transport and phosphorylation rates is achieved only at high glucose concentrations (400 to 500 mg %). In further increase in glucose concentration, phosphorylation becomes a limiting reaction [2]. In other words, glucose transport rate from intercellular medium through cytoplasmic membrane into intracellular medium is a process limiting glucose utilization rate by a live tissue.

Originating from the above consideration, it appears logical and completely reasoned to draw a conclusion that heat production as well as glucose utilization rate is a linear function of blood glucose concentration and measurement of local heat production value allows for determining blood glucose level.

The Method of Micro Calorimetry of Local Metabolism Heat Effect

Water flow density determining intensity of steam cooling equals difference between heat production by a tissue and heat exchange determined by radiant radiation, heat conductivity and convection:

E=M−R−T−C

Heat production may be expressed as follows:

M=E+a*(Tₛₖᵢₙ−T)

The latter ratio interrelating local metabolism rate, evaporative cooling intensity and heat exchange caused by temperature difference between body surface and air, allows for determining heat production value by measuring water flow density through the epidermal corneous layer and ambient air temperature.

M=Eₚᵣₑₛₛᵤᵣₑ+E_{mat.}+a*(Tₛₖᵢₙ−T₀)+a*(T₀−T)

In the patent [19,22] correlation between blood sugar level and skin surface temperature has been established.

B*M=a*(Tₛₖᵢₙ−T₀)

The expression attains the following form:

M−a*(Tₛₖᵢₙ−T₀)=(1−b)*M=Eₚᵣₑₛₛᵤᵣₑ+E_{mat.}+a*(T₀−T).

The expression finally has the following form:

(1−b)*M=Eₚᵣₑₛₛᵤᵣₑ+E_{mat.}+a*(T₀−T)=E_{exp.}+a*(T₀−T)

Here the following designations are accepted:

Tₛₖᵢₙ is body surface temperature

T₀ is air temperature at which intensity of evaporative cooling process equals to zero.

T is ambient air temperature.

Eₚᵣₑₛₛᵤᵣₑ is flow density of water transport of which is caused by external pressure to the body surface.

Eₘₐₜᵤᵣₑ+αis flow density of water transport of which is caused by natural process of non-perceived perspiration.

A, b are constants.

As a result of the experimental studies carried out by the inventors, linear relationship between water flow density through ECL and ambient temperature, external pressure onto the body surface and blood sugar concentration has been established.

Elevation of ambient temperature results in a lineally proportional increase in water flow density through ECL. At the same time, rise in heat exchange due to increase in evaporative cooling intensity is exactly equal to reduction in heat exchange caused by temperature difference between the body surface and the environment.

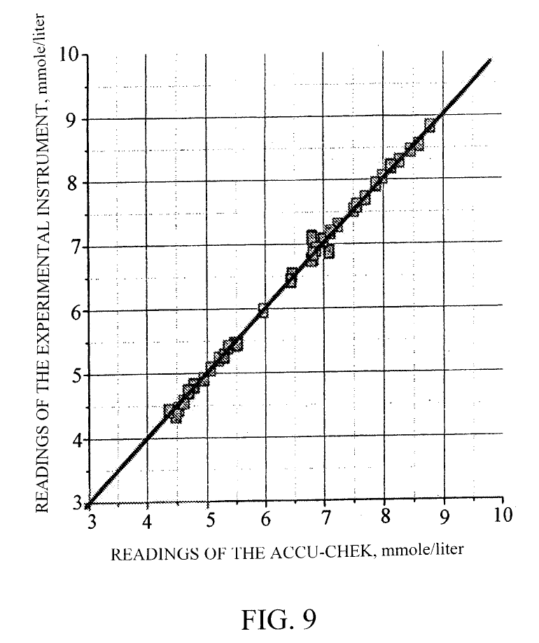

Similarly, increase in blood sugar level results in a lineally proportional increase in water flow density through the ECL and as a sequence, in a proportional growth of heat exchange caused by evaporative cooling. In a constant ambient temperature, increase in heat exchange due to evaporative cooling caused by increase in blood sugar level is exactly equal to increase in heat power of cellular tissue metabolism (heat production of tissue). Typical experimental results are presented in FIGS. 22, 23, 9, 32.

The experimental results obtained, directly indicate to the diffuse transfer mechanism of heat generated during cellular glucose metabolism to the body surface. This mechanism has the following typical peculiarities:

1. A resulting trans-capillary water flow through intercellular space is transferred to the body surface and maintains evaporative cooling process. A value of a resulting trans-capillary water flow is lineally proportionally dependent on blood glucose concentration and ambient temperature.

2. Heat generated during cellular metabolism is absorbed by intercellular water flow due to a high heat capacity thereof, it is transferred from deep layers to the body surface and maintains balance of a tissue heat exchange with the environment. A value of heat power (heat production) of cellular metabolism is lineally proportionally dependent on blood glucose concentration.

3. A value of a resulting trans-capillary water flow, evaporative cooling intensity as well as glucose utilization and heat production rates are linear functions of blood glucose concentration.

In other words, evaporative cooling intensity including a non-diffuse heat transfer from depth to surface (emission of heat generated in a cell to surface) and intensity of cellular heat generation process (heat production) are determined by blood glucose concentration. A rate of the both processes is lineally dependent on blood glucose concentration and as a sequence, power of evaporative cooling process is equal to heat production power minus power of external heat flow determined by ambient temperature. This mechanism supports constancy of a live tissue temperature and provides for an extremely high stability of temperature.

The results obtained by the inventors, directly indicate to the fact that intercellular substance is in fact a specific natural isothermal micro calorimeter of heat power providing for a local heat balance of a tissue:

The power of evaporative cooling is equal to heat power of metabolism minus power of heat flow of heat exchange caused by difference of temperature.

Thus, measuring a value of heat power of local metabolism (heat production) comes to measuring water flow density through the epidermal corneous layer and ambient temperature. Such measurement method allows for unequivocal determination of blood sugar level, since a rate of tissue sugar absorption and as a sequence heat production are synonymous functions of blood sugar level.

In the following section “Biophysical fundamentals: physics of intercellular substance”, physical mechanisms determining a lineally proportional relationship between pressure in the microcirculation system, resulting trans-capillary flow, density of water flow through the epidermis on one hand and blood sugar concentration on other hand are given consideration.

### Biophysical Fundamentals

### Physics of Intercellular Substance

In the section “A method of micro calorimetry of local metabolism thermal effect”, there is given consideration to the experimental results that directly indicate to the fact that intercellular substance is a specific natural isothermal micro calorimeter of heat power, for which the following ratio is fulfilled: power of evaporative cooling=heat power of metabolism—power of heat flow of heat exchange caused by difference of temperature.

This ratio interrelating power of evaporative cooling, heat power of metabolism and power of heat flow of heat exchange caused by difference of temperature, is in fact a condition providing for constancy of tissue temperature.

Taking into consideration that density of water flow through a tissue surface during a non-perceivable perspiration is a value determined by a resulting trans-capillary water flow dependent on a value of average capillary pressure and intensity of cellular metabolism is a function of blood glucose concentration, the latter expression given consideration in the previous section, is transformed into the following form:

P=F(C,T),

wherein

P is a mean value of capillary pressure,

C is blood sugar level,

T is air temperature.

In accordance with this ratio, capillary pressure is a function of blood sugar concentration and air temperature.

The experimental studies carried out by the inventors, have supported equitability of the latter ratio that is in fact a direct sequence of leveling heat balance providing for constancy of the body temperature.

In order to comprehend physical mechanisms and to explain relationship between capillary pressure on one hand, and temperature and blood glucose concentration on the other hand obtained experimentally, physical properties of intercellular substance have been theoretically substantiated.

The theoretical study has been carried out within the frames of a physical model system taking into consideration peculiarities of the intercellular substance molecular structure as a long polymeric molecular chain, and giving consideration to the intercellular substance as to a system consisting of a large number of interacting particles. Behavior of such system has been investigated near the stability border determined by the ordering temperature which in energetic units, is by the value order of equal to the typical energy of interaction between the system particles.

Within the frames of such model, the inventors has managed to obtain an exact solution for the energy of intermolecular interaction and to obtain exact analytic expressions for tissue pressure (osmotic pressure of intercellular substance) and elastic strain of intercellular substance (elastic pressure) depending on variables of the intercellular substance states, i.e. blood glucose concentration, external pressure and temperature.

Further the results of the study carried out by the inventors, are used in the text without explanation of the methods using which they have been obtained. In particular, the diagrams of analytical functions of tissue (osmotic) pressure and elastic strain of the intercellular substance depending on variables of the state, are presented and used here without giving consideration to analytic expression of the functions as such.

The study of the system behavior depending on the following state variables has been carried out: temperature (T), pressure (P), concentration of biochemical ingredients in blood, glucose concentration (C).

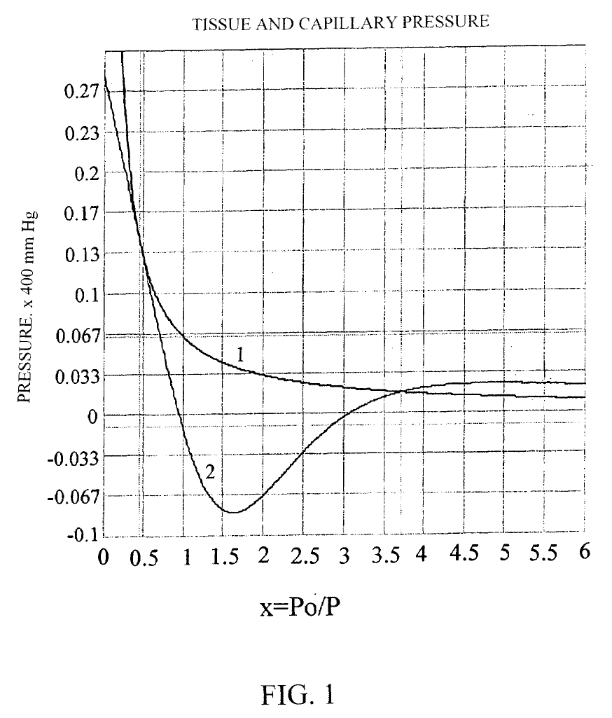

In FIG. 1, diagrams of relationship between osmotic pressure of intercellular substance and capillary pressure and the non-dimensional parameter α(═P₀/P are presented, wherein P is the variable (pressure within a capillary), P₀ is average capillary pressure.

The curve 1 (the blue curve) is a diagram of relationship between capillary pressure and the “α” parameter. The curve 2 (the red curve) is a diagram of relationship between tissue pressure and the “α” parameter.

The diagrams have two common points: “a” (the arterial end of a capillary) is a point of touching the two diagrams; “b” (the venous end of a capillary) is a point of intersection of the two diagrams. Intra-capillary pressure in the points “a” and “b” are equal to the tissue pressure (osmotic pressure of intercellular substance). Within the interval of external pressures [a, 1] (the region of high pressures) tissue pressure attains positive values. Within this range of pressures, swelling basic substance and distension of intercellular substance (increase in volume) occurs. Within the interval of external pressures [1,3] tissue pressure attains negative values. Within this range of external pressures, dehydration and compression of intercellular substance (diminishing volume) occurs.

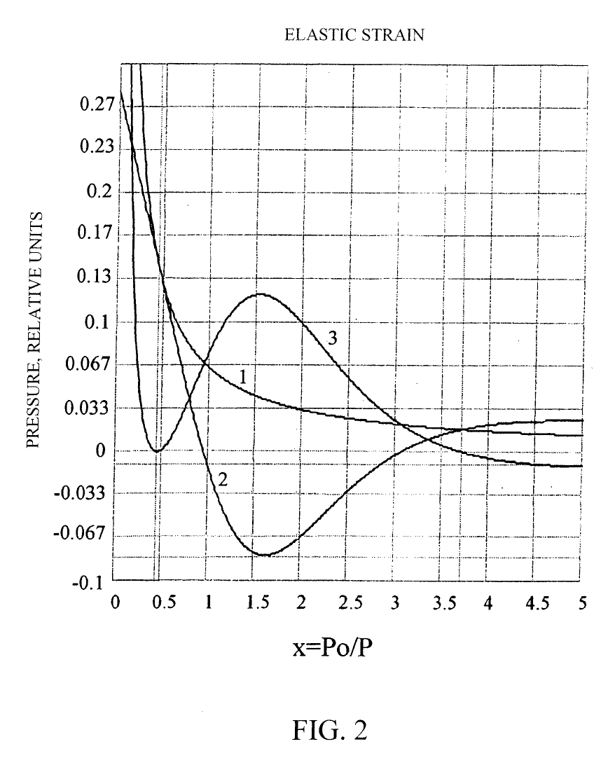

Within this range of external pressures [3, b] (a region of low pressures) tissue pressure attains positive values. Within this range of pressures, swelling basic substance and distension of intercellular substance occurs. Swelling degree of the intercellular substance is determined by an amount of water in the intercellular substance volume. The special points wherein intra-capillary pressure is equal to the intercellular substance pressure, determine the range of intra-capillary pressures between inlet and outlet thereof. The point “b” determines the value of a minimum (outlet) hydraulic intra-capillary pressure and the point “a” is the value of the maximum pressure or inlet capillary pressure. Such character of relationship between intercellular substance tissue pressure and external pressure value (at a fixed value of glucose concentration) results in occurrence of heterogenous distribution of elastic strain (elastic pressure) along blood vessels and in particular capillaries. In FIG. 2, relationship between elastic strain of intercellular substance and hydraulic pressure in a blood vessel is presented.

Relationship between elastic strain of intercellular substance and hydraulic intra-capillary pressure value has the following typical characteristics:

1. Difference between capillary and tissue pressures is equilibrated by elastic pressure (elastic strain of intercellular substance). In this sense, a capillary is not a tube a resilient envelope of which equilibrates intra-capillary pressure but it presents a tunnel in the intercellular substance elastic strain And tissue pressure of which equilibrate intra-capillary pressure.

2. A non-linear dependency character of elastic strain around the point “a” (inlet of a capillary) results in formation of narrowing of a “bottle neck” type. Capillary lumen increases in the direction of the venous end thereof, in spite of reduction in intra-capillary hydraulic pressure. Such narrowing exerts a main hydraulic resistance to a flow through a capillary, it determines throughput thereof and results in a significant fall of hydraulic pressure in the initial capillary site.

3. The region of high (arterial) pressures is located at the left from the point “a” and the region of low (venous) pressures is located at the right from the point “b”.

4. Mechanical equilibrium of a capillary envelope (the tunnel wall) is determined by equilibrium between intra-capillary hydraulic pressure and osmotic and elastic pressure of intercellular substance.

The condition of the mechanical equilibrium in the point “b” has the following form:

Tissue pressure (osmotic pressure)=Hydraulic intra-capillary pressure. Elastic strain (elastic pressure)=zero.

Change in blood sugar level results in disorder of mechanical equilibrium and occurrence of elastic strain which is not counterbalanced by intra-capillary hydraulic pressure.

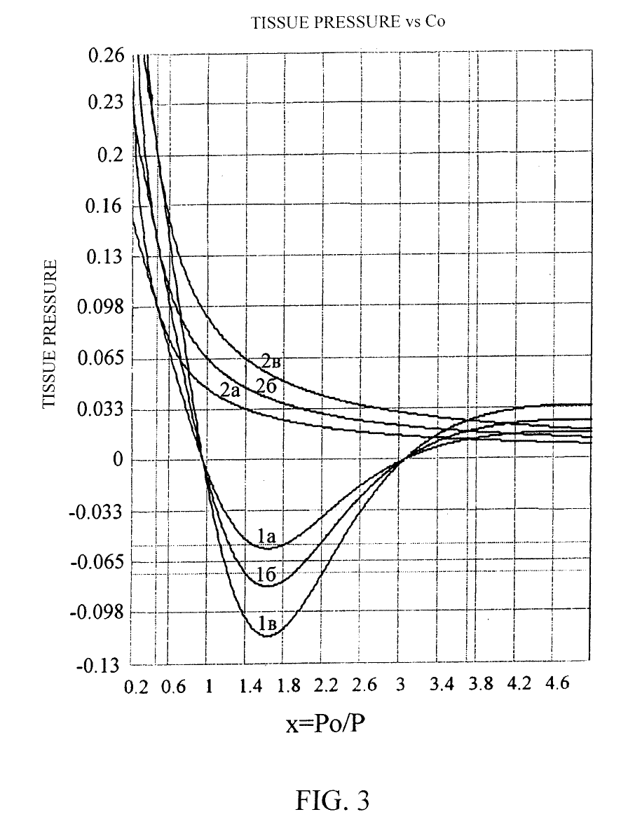

At the same time, increase in swelling degree of intercellular substance, reduction in a capillary lumen (cross-section) in the “a” point, increase in resistance to blood flow occur and as a sequence, lowering pressure in an initial capillary portion and elevation of capillary inlet pressure (in the “a” point). Mechanical equilibrium is established after leveling inlet tissue and capillary pressure. This process results in change in equilibrium distributions of hydraulic intra-capillary pressure and elastic pressure of intercellular substance toward the venous capillary end. Establishment of mechanical equilibrium in the “a” point results in establishment of equilibrium along all capillary length. FIG. 3 shows diagrams of relationship between equilibrium distributions of tissue (curves 1) and capillary (curves 2) pressures depending on the “α” parameter for different values of blood sugar level.

The characteristic feature of the obtained relationships consists in that in elevation of blood sugar level, position of the points wherein elastic strain of intercellular substance is equal to zero (the points “a” and “b”) on the abscissa axis remains unchanged. This means that a proportional elevation of intra-capillary pressure in all points occurs over a length from capillary inlet to outlet. Inlet pressure (the maximum pressure in the system) and outlet pressure (the minimum pressure in the system) as well as pressure in any other point inside a capillary are linear functions of blood sugar level and at the same time, the ratio Pₘₐₓ/Pₘᵢₙ=Pₐ/P_{b}=3.72/0.46=8.087 remains constant.

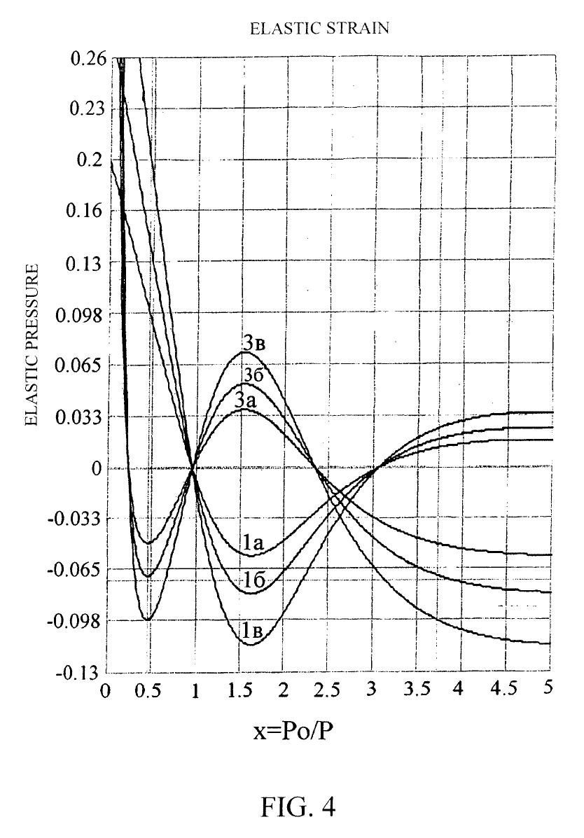

FIG. 4 presents diagrams of equilibrium distribution of relationship between elastic pressure of intercellular substance and hydraulic pressure at different values of blood sugar.

The diagrams presented in FIG. 4, allow for understanding the nature and mechanisms of relationship between hydraulic pressure in the cardiovascular system and blood sugar level: rise in blood sugar level results in increased swelling within the interval of “α” values [0.25, 1] and narrowing capillary lumen in the “a” point. Similarly, capillary lumen in the “b” point diminishes. Arterial and venous resistance determining hydraulic resistance of the blood circulation system, are linear functions of blood sugar level (within the range of control thereof).

Along with blood sugar level a linear proportional growth of arterial and venous pressure occurs, pressure drop in a capillary grows and arterial pressure rises. At the same time, volume flow through the capillary remains constant.

This mechanism also allows for explaining constancy of volume flow of the tissue liquid circulating in intercellular space (the microcirculation flow) and delivering sugars to tissue cells and removal of metabolism products.

To an equal degree, the mechanism given consideration allows for explaining water transportation from deep layers to the body surface. Water delivery rate from a capillary vessel into intercellular space is determined by a value of resulting trans-capillary flow.

Water flow from depth to surface provides for transferring heat generated during cellular metabolism, maintains steam cooling process and shows linear proportional relationship with blood sugar level and air temperature.

The presented relationships have peculiarities in the ponts “α=1” and “α(=0.25”: elastic pressure in these points is equal to capillary pressure of zero flow. Elastic pressure in the interval between these points is less than capillary pressure of zero flow and it is equal to zero in the point “α(=0.46”.

In glucose concentration equal to 4.5 mmole/liter, hydraulic pressure values are respectively equal to the following values:

25 mm Hg−in the point “α=1” (capillary pressure);

54.3 mm Hg−in the point “α=0.46” (inlet capillary pressure);

100 mm Hg−in the point “α=0.25” (average arterial pressure);

6.7 mm Hg−in the point “αt=3.72” (outlet capillary pressure).

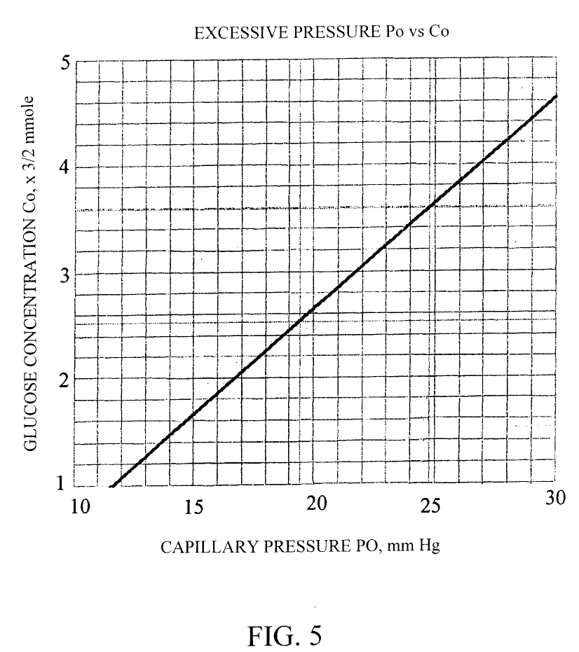

FIG. 5 shows a diagram of relationship between average capillary pressure and blood sugar level.

Capillary pressure corresponding to the pressure of zer flow, is numerically equal to plasma oncotic pressure value and therefore, in elevation of blood sugar level and rise in average capillary pressure, a shift of the zero flow point toward venous end of a capillary occurs. Such shift of the zero flow point results in increased filtration area, rise in filtration flow and increase in a resulting trans-capillary flow which also appears to be a linear function of blood sugar level.

The inventors within the frames of the selected physical model have also managed to obtain exact expressions for the relationship between capillary pressure and resulting trans-capillary flow on one hand, and air temperature on the other hand.

Thus, the inventors within the frames of a simple but at the same time stringent physical model have managed to obtain exact expressions for the relationship between main parameters of microcirculation and metabolism on one hand, and blood sugar level on the other hand and to explain the self-regulation phenomenon in the microcirculation system.

Biophysical Fundamentals: The Mechanism of Tissue Fluid Transport in intercellular space

The physical characteristics of intercellular substance given consideration above, also allow for explaining the mechanism of tissue fluid transport in intercellular space. Typical distance between surfaces of adjacent cells is known to have a value of a micrometer order. Tissue fluid from a capillary wall to a cell, is obviously transported along channels a lumen of which is less than a typical intercellular distance.

The physical characteristics of intercellular substance given consideration above, allow for explaining the mechanism of fluid transport in intercellular space.

Heterogenic distribution of osmotic pressure of intercellular substance along a capillary vessel (FIG. 4) results in heterogenic distribution of osmotic and elastic pressures in the tissue volume. Specificity of heterogenic volume distribution of the pressures consists in the presence of pressure (hydraulic, osmotic and elastic one) drops in intercellular substance between arterial and venous end of capillary vessels. Pressure gradients are generated between the both adjacent capillaries and within one capillary. Such pressure gradients result in formation in intercellular substance of narrow channels oriented by the pressure gradient, which channels begin in the arterial region of a capillary and end in the venous region. Intercellular fluid is transported by these channels which are specific “micro capillaries”. Difference of hydraulic pressures is a motive force of tissue fluid volume flow through such “micro capillary”. At the same time, distribution of tissue pressure along such channels depending on intra-channel hydraulic pressure obeys the same regularities which describe distribution of pressures in a capillary vessel. These regularities have been given consideration above in the section “Biophysical fundamentals: physics of intercellular substance” (FIG. 4).

A typical peculiarity of the intercellular substance characteristics given consideration above is that volume flow of the tissue fluid circulating in intercellular space remains constant in fluctuations of hydraulic pressure in the microcirculation system. Linear relationship between glucose absorption rate and heat production on one hand and blood sugar concentration on other hand is a sequence of the mentioned peculiarity, since glucose flow density from a capillary to a cell is determined by product of volume flow of intercellular substance fluid by blood sugar concentration.

Biophysical Fundamentals: The Mechanism of Water Transport Through the Epidermis During Non-Perceived Perspiration

Under natural conditions, distribution of intercellular substance (osmotic) pressure is non-uniform. Osmotic pressure of intercellular substance located adjacently to capillaries is determined by blood sugar level. With advancement from deep layers (the dermal papillary layer) to the epidermis superficial layers (epidermal corneous layer), tissue pressure lowers down to zero. Lowering intercellular substance pressure down to zero is a result of that external pressure to the epidermal corneous layer surface is equal to atmospheric pressure. Relationship between osmotic pressure of intercellular substance and external pressure presented in FIGS. 1-4 and within the range of pressures [0.1] is lineally proportional. Along with increase in a value of average intra-capillary hydraulic pressure, lineally proportional elevation of osmotic pressure in the intercellular substance surrounding a capillary occurs. Osmotic pressure gradient by the epidermis thickness that proves to be equal to the difference between an average value of capillary pressure and the pressure of zero flow, results in hydraulic pressure gradient of tissue fluid. Hydraulic pressure gradient is a motive force of tissue fluid volume flow through the epidermis, the value of this flow proving to be equal to resulting trans-capillary flow. In other words, water flow density through the epidermis (intensity of steam cooling process), resulting trans-capillary flow and intra-capillary hydraulic pressure interrelate by the ratio:

P_{excessive}=P_{average}−P_{zero flow}=J_{resulting}=J_{ECL}

Biophysical Fundamentals: The Mechanism of a Non-Diffusion Heat Transfer from Depth to Surface

Under normal physiologic conditions, the temperature of internal tissues (37° C.) is as a rule higher than the temperature of superficial tissues (30° C.). Temperature is a variable of the intercellular substance condition and therefore, temperature difference between two spatially divided points, results in osmotic pressure gradient of intercellular substance and hydraulic pressure of tissue fluid between these points. Hydraulic pressure of tissue fluid rises with elevation of tissue temperature. Temperature gradient directed from depth to surface results in pressure gradient which is a motive force of tissue fluid volume flow by intercellular space from depth to surface. This process provides for transfer of heat generated as a result of cellular metabolism from depth to surface and concurrently maintains the steam cooling process (a non-perceived perspiration). Heat generated during cellular metabolism is absorbed by tissue fluid because of a high heat capacity of water, is transported by the intercellular space to the body surface and scattered into the environment by steam cooling.

Thus, the mechanism of heat transfer process is non-diffusion one. Difference between hydraulic pressures of tissue fluid and not difference of temperature is a motive force of the process. Water (tissue fluid) circulating from depth to surface by intercellular space transfers the heat generated resulting from cellular tissue metabolism.

Biophysical Fundamentals: The Mechanism of Cardiac and Vascular Self-Regulation

Change in power of the heart ventricular contraction is known to be directly proportional to an average blood pressure value (BP) [N. M. Amosov et al., (1969)]. Constancy of stroke volume and cardiac output is an essential characteristic of this relationship. The described relationship between cardiac contraction power and average aortic pressure is observed in a rather broad but limited range of BP change (approximately from 40-50 to 130-150 mm Hg). In exit beyond these limits, BP effect on contraction energy becomes diametrically opposite. Irrespectively of venous pressure, BP regulates ventricular contraction power. Power generated by the heart changes as effected by BP exactly to the degree which is needed to provide for constancy of cardiac output. Due to this, the heart is capable of regulating contraction power thereof within wide limits preserving a stroke volume predetermined by blood inflow.

Starling in his classical works (1914, 1918) had for the first time indicated to a direct relationship between cardiac contraction power and arterial resistance and venous inflow.

The described biophysical mechanism of self-regulation in the microcirculation system establishing a direct relationship between hydraulic resistance and pressure in the microcirculation system on one hand and blood sugar level, temperature and external pressure on the other hand, allows for explaining a nature of the phenomenon known as self-regulation of the heart and vessels. In fact, change in hydraulic resistance of capillary vessels occurring in change in blood sugar level (in a constant ambient temperature and atmospheric pressure), results in change in pressure drop between inlet and outlet of a capillary vessel and in change in blood pressure. Changes in blood pressure in their turn lead to change in cardiac contraction power in such way that stroke volume and cardiac output are maintained at a constant level.

Thus, change in blood sugar level results in lineally proportional changes in pressure in the blood circulation system, i.e. average capillary pressure, pressure in arterial and venous ends of a capillary, blood pressure and venous pressure are all changed. Moreover, distribution of hydraulic pressure in the blood circulation system is an unequivocal function of the blood biochemical composition, in particular, blood sugar level.

### A Method for Determining Amount of Water in Intercellular Substance and Water Flow Density Through the Epidermis

The method consists in a time course of intercellular substance swelling process in applying (with a dosed pressure) on the epidermal corneous layer a water-impermeable applicator excluding evaporation of water from a local surface.

Water content in intercellular substance and a value of resulting trans-capillary water flow through the epidermis can be determined using a method the essence of which consists in a continuous measurement of a time course of water amount in intercellular substance in a tissue volume located under the water-impermeable applicator. One of practical methods allowing for determining water amount in the intercellular substance, is a method which allows for determining water amount in the intercellular substance by measuring a time course of water amount in the superficial epidermal corneous layer (ECL). This method allows for determining dynamics of water content and equilibrium content thereof in the intercellular space of deep dermal layers and subcutaneous tissues, by a character of a time course of water amount (weight) in the ECL.

The water-impermeable applicator which is applied onto the ECL surface with a dosed pressure, excludes the possibility for natural evaporation of water from the ECL surface during a non-perceived perspiration. This results in disturbance of a natural balance between a resulting trans-capillary water flow, water flow delivered to the epidermal surface from dermal layer, wherein a capillary network is located and water flow evaporating from the ECL surface. Disturbance of a natural balance of the flows results in occurrence of local swelling process of the intercellular substance in a tissue volume under the applicator.

Under natural conditions, osmotic pressure distribution in the intercellular substance is non-uniform. Osmotic pressure of the intercellular substance located adjacently to a blood capillary is determined by blood sugar level. With advancement from deep layers (the papillary dermal layer) to the epidermal superficial layers (the epidermal corneous layer), lowering tissue (osmotic) pressure value down to zero occurs. Lowering the intercellular substance pressure down to zero is a sequence of the fact that external pressure onto the epidermal corneous layer surface is equal to atmospheric pressure. Zero level of tissue pressure corresponds to atmospheric pressure.

As swelling of the intercellular substance progresses, leveling osmotic pressure of the intercellular substance throughout the epidermis thickness occurs. Leveling osmotic pressure with time results in a gradual diminishing a value of water flow density through the epidermis and trans-capillary water flow to zero.

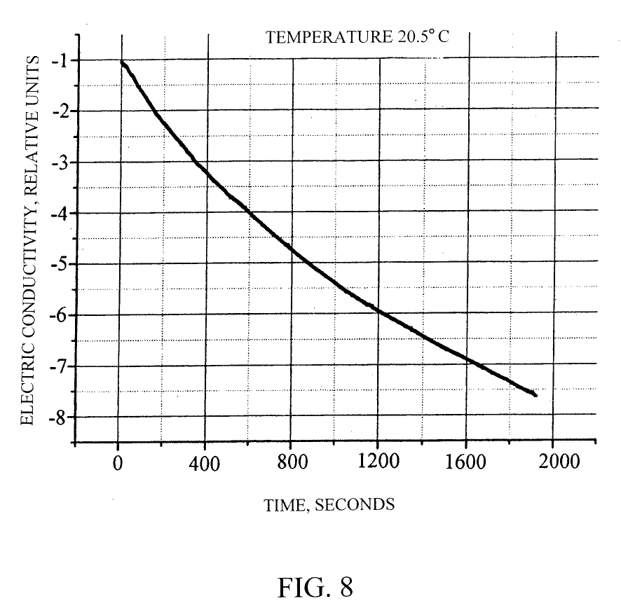

FIG. 8 shows a typical time course of swelling the intercellular substance of the controlled tissue site, arising following application to the ECL surface of the water-impermeable applicator excluding evaporation of water form the surface of the controlled body site.

Under the conditions of a non-stationary process of swelling the intercellular substance, water flow density through the epidermis J(t) and amount (mass) of water in the superficial corneous layer of the epidermis m_{ecl} are interrelated by the following differential equation:

J(t)=F(m_{ecl}dm_{ecl}/dt,d²m_{ecl}/dt²)

wherein m_{ecl} is water mass in the controlled ECL volume at the time moment t.

Such method for determining water flow density through the ECL, is based on the fact that water flow density through the epidermis is equal to a resulting trans-capillary flow which in his turn, is equal (with accuracy up to a constant coefficient) to an excessive hydraulic intracapillary pressure (this has been given consideration in the previous section):

P_{excessive}=P_{average}−P_{zero flow}=J_{resulting}=J_{ECL}

Excessive hydraulic intra-capillary pressure and tissue pressure are interrelated with the aid of the following similar second order differential equation:

P_{excessive}(t)=F(PₜᵢₛₛᵤₑdPₜᵢₛₛᵤₑ/dt,d²Pₜᵢₛₛᵤₑ/dt²)

wherein Pₜᵢₛₛᵤₑ(t) is a tissue (osmotic) pressure as a function of time.

The expression for equilibrium value of water content in the intercellular substance (ICS) of the dermal skin layer (the skin layer wherein skin capillary network is located) has the following form:

M_{ics}(t)=F(m_{ecl}d_{mecl}/dt,d²m_{ecl}/dt²).

This differential equation establishes relationship between water content in the intercellular substance of the capillary dermal layer (the papillary layer) and water content in the superficial epidermal corneous layer.

The physical mechanisms determining functional relationship between intra-capillary hydraulic pressure, trans-capillary flow, osmotic pressure, water content in the intercellular substance and blood sugar content, have been given consideration above in the section “Biophysical fundamentals: Physics of the intercellular substance”.

### A Method for Measuring Local Tissue Metabolism Rate

The method for determining local tissue metabolism rate by measurement of air temperature and the rate of steam cooling process determined by water transport rate through the ECL is described in the section “The micro calorimetry method of local metabolism's thermal effect”.

In the previous section (“A method for determining amount of water in intercellular substance and water flow density through the epidermis”), the method for determining a resulting trans-capillary flow and water flow density through the ECL is described which method is based on measuring water amount in the intercellular substance. Such method makes it possible to measure a tissue local metabolism rate determined by sugar absorption rate by the tissue, by measuring air temperature and water amount in the intercellular substance.

The method for measuring blood sugar level is based on the measurement of a tissue local metabolism rate using the method described above.

The method for measuring local metabolism rate (sugar absorption rate by a tissue) makes it possible to determine sensitivity of the tissue to insulin and to early diagnose type 2 diabetes.

### A Method for Determining Average Capillary Pressure

The equation establishing interrelations between a value of intra capillary hydraulic pressure and a value of tissue pressure and water content in thew intercellular substance has the following form:

P_{capillary}(t)=F(PₜᵢₛₛᵤₑdPₜᵢₛₛᵤₑ/dt,d²Pₜᵢₛₛᵤₑ/dt²)=F(m_{ecl} dm_{ecl}/d t,d²m_{ecl}/dt²)

Calibration is performed by tissue pressure (water content in the intercellular substance) as a function of external pressure onto the surface of a controllable local site.

### A Method for Determining Average Blood Pressure

The equation establishing interrelations between a value of average blood pressure and a value of tissue pressure and water content in the intercellular substance has the following form:

P_{blood}(t)=F(PₜᵢₛₛᵤₑdPₜᵢₛₛᵤₑ/dt,d²Pₜᵢₛₛᵤₑ/dt²)=F(m_{ecl}dm_{ecl}/dt, dm_{ecl}/dt²)

Calibration is performed by m_{ics} as a function of Pₑₓₜₑᵣₙₐₗ,

Pₑₓₜₑᵣₙₐₗ is external excessive pressure on the body surface.

### A Method for Determining Blood Level of Biochemical Ingredients by Level Thereof in the Epidermal Corneous Layer

Flow density of a biochemical ingredient is determined using a continuous recording time course of mass transfer of this ingredient by level thereof in the ECL and using determining derivatives of time course.

The expression for flow density of a biochemical ingredient and mass of this ingredient in the epidermal corneous layer are interrelated by a second order differential equation and it has the following form:

J_{xecl}(t)=F(m_{xecl}dm_{xecl}/dt,d²mx_{ecl}/dt²)

wherein is a biochemical ingredient mass in the controlled volume of the ECL at the time moment t.

Flow density of a biochemical ingredient determined using such method is a linear function of blood level of this ingredient. Level of a biochemical ingredient in the epidermal corneous layer is determined using an electrochemical probe or by any other possible method.

Blood level of a biochemical ingredient and level of this ingredient in the epidermal corneous layer are interrelated by the following equation:

mx_{ecl}(t)=F(m_{xecl}dm_{xecl}/dt,d²m_{xecl}/dt²)

An individual case of the method for measuring blood level of a biochemical ingredient described above is a method for measuring blood sugar level by level thereof in the epidermal corneous layer.

The expression for glucose flow density and glucose mass in the epidermal corneous layer are interrelated by the differential equation having the following form:

J_{g}(t)=F(m_{gecl}dm_{gecl}/dt,d²m_{gecl}/dt²)

wherein m_{g} is glucose mass in the controlled volume of the ECL at the time moment t. Glucose flow density is a linear function of blood sugar level. Glucose level in the epidermal corneous layer is determined using a standard electrochemical probe or using any other probe or method allowing for determining glucose level in the corneous layer.

Blood sugar level and sugar level in the epidermal corneous layer are interrelated by the equation:

M_{ics}(t)=F(m_{gecl}dm_{gecl}/dt,d²mg_{ecl}/dt²).

### The Electrometric Method for Measuring Water Amount in the Intercellular substance

The method for measuring water amount in the intercellular substance by water level the epidermal corneous layer has been given consideration in the section “A method for measuring water amount in the intercellular substance”. In the instant section, description of the electrochemical method for measuring water content in the intercellular substance.

The method is based on the results experimentally obtained by the Inventors:

1) transverse electric conductivity of the ECL is a parameter depending on water content in the corneous layer and measurement of transverse electric conductivity of the ECL allows for determining water amount in this layer with a high accuracy;

2) time course of transverse electric conductivity of the ECL measurable using a dry, flat and water-impermeable electrode, is a sequence of time course of water amount in the corneous layer and measuring time course of transverse electric conductivity of the ECL allows for determining water content in the intercellular substance of deep layers.

Water flow density through the epidermis and transverse electric conductivity of the epidermal corneous layer are interrelated by the differential equation having the following form:

J(t)=F(δ(t),dδ/dt,d²δ/dt²)

wherein δ(t) is transverse electric conductivity of the ECL, J(t) is water flow density through the ECL.

Water amount in the intercellular substance m_{ics}(t) of the skin dermal layer and transverse electric conductivity of the epidermal corneous layer are related by the similar equation:

m_{ics}(t)=F(δ(t),dδ/dt,d²δ/dt²).

The values of hydraulic capillary pressure and the resulting trans capillary water flow are interrelated with transverse electric conductivity of the ECL by similar equations.

Thus, a continuous measurement of time course of transverse electric conductivity of the ECL allows for determining in the continuous measurement regimen, water amount in the intercellular substance, a value of intra-capillary hydraulic pressure as well as a value of a resulting trans-capillary water flow and water flow density through the epidermis.

The proposed method can be realized using a device for measuring electric characteristics of the epidermal corneous layer described in the works [6,7].

Essence of the method consists in measuring transverse electric conductivity of the superficial epidermal corneous layer using a dry, water-impermeable electrode applied to the skin surface of the body using a dosed pressure.

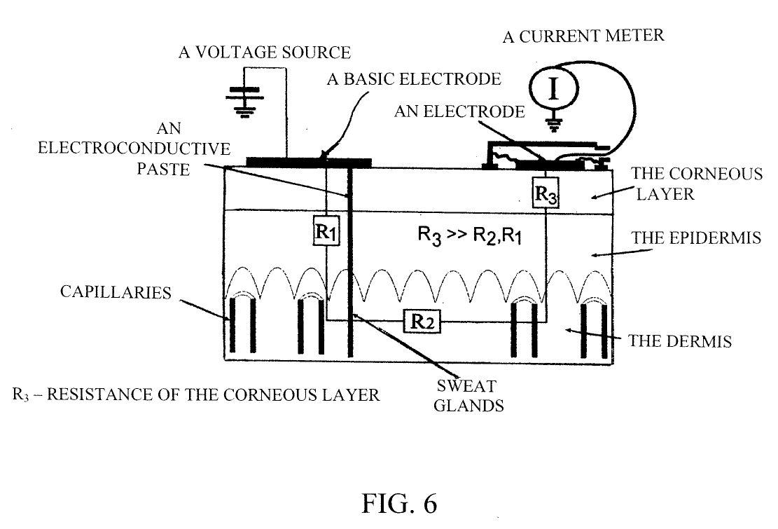

The equivalent electric circuit of the device using which the electrometric method of measurement described above is practiced, is depicted in FIG. 6.

The device consists of a base electrode 1, applicable to the skin surface 2 through a layer of a conductive material 3 allowing for providing electric contact with the skin ( ) in fact, liquids, emulsions and pastes having a high conductivity are used) as well as a measuring electrode 4 applicable directly to the skin surface 2. The measuring electrode has a flat surface and it is manufactured of a conductive, water-impermeable material.

The base electrode 1 is connected with a common bar via a voltage source 5. The measuring electrode is connected with a common bar via a measuring unit 6.

The device operates in the following way. Following application of voltage in the circuit: the base electrode—the skin—the measuring electrode—the measuring unit—the voltage source, current runs therein which current is dependent on transverse electric conductivity value of the superficial epidermal corneous layer onto which the measuring electrode 4 is applied. By measuring a value of current and time course thereof using the measuring unit 6, a value of transverse electric conductivity value of the epidermal corneous layer is determined.

In the given scheme of measurement, due to use of a conductive paste, electric resistance R₁ lowers down to the values 100 kOhm/cm² and becomes of the same order as electric resistance R₂ of internal tissues. As a result, the values of resistance R₁ and R₂ may be neglected as compared with resistance R₃ and electric current in the measuring circuit is determined only by resistance R₃ which as a rule is of 1 gOhm/cm². Measurable current is practically determined by electric resistance of the skin site's corneous layer under the measuring electrode. Electric impedance measured using such method, is unequivocally related to water content in the corneous layer and time course thereof is unequivocally determined by swelling time course of the intercellular substance (a volume of intercellular space determined by water content in the intercellular substance).

FIG. 8 shows a typical time course of transverse electric conductivity of the epidermal corneous layer measurable using the method described above.

The flat, water-impermeable measuring electrode secured on the corneous layer surface excludes the possibility of water evaporation from the surface thereof during a non-perceived perspiration and results in disturbance of a natural balance between the flow of water evaporating from the ECL surface and a resulting capillary flow. Such disturbance of a local natural balance results in swelling process of the intercellular substance. Time course of swelling process of the intercellular substance is recorded by time course of transverse electric resistance of the epidermal corneous layer. Increase in water amount in the intercellular space results in increased amount thereof in the corneous layer that results in increase in electric conductivity of the superficial epidermal layer. Typical time course of transverse electric resistance measurable using such method is presented in FIG. 8. Under natural conditions in absent measuring electrode on the body surface these flows are equilibrated and provide for transfer of heat generated during cellular metabolism from deep layers to the body surface. The physical mechanism of water and heat transfer processes from depth to surface has been given consideration in the sections “Biophysical fundamentals: the mechanism of water transport through the epidermis” and “Biophysical fundamentals: the mechanism of a non-diffusion heat transfer from depth to surface”.

Thus, measuring time course of swelling using measurement of transverse conductivity time course, allows for determining values of the following parameters of a local tissue: water content in the intercellular substance, an average value of capillary pressure, osmotic pressure of capillary pressure, a resulting trans-capillary flow, a value of tissue heat production in a tissue volume under the electrode.

### A Method for Measuring Blood Sugar Level

The method for measuring blood sugar level based on micro calorimetric measurement of a local heat production, is described in the section “A method of micro calorimetry of local metabolism”. The method is based on measuring a local heat production of a tissue using measurement of ambient temperature and a rate of steam cooling process determined by water flow density through the epidermis. The method of measuring local metabolism rate is described in the section “A method of measuring local metabolism rate”.

A method of determining water flow density through the epidermis which is based on measuring water content in the intercellular substance, is described in the sections “A method of measuring water content in the intercellular substance” and “The electrometric method for measuring water amount in the intercellular substance”.

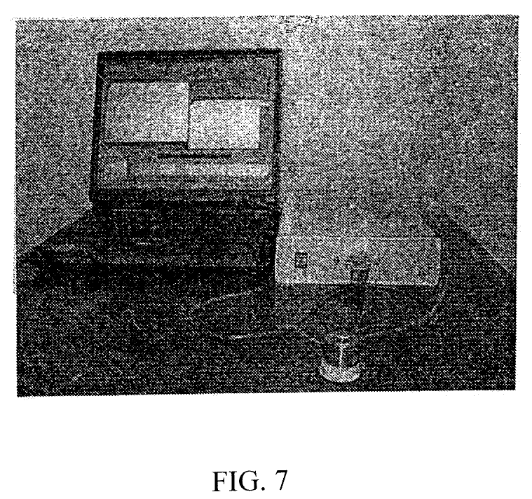

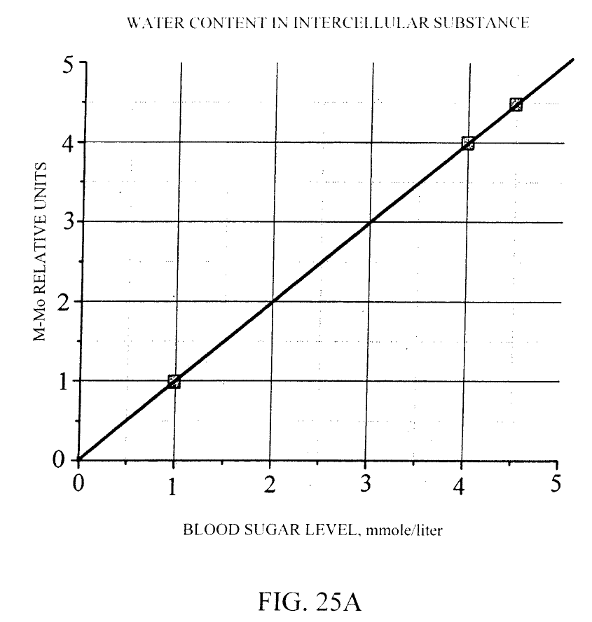

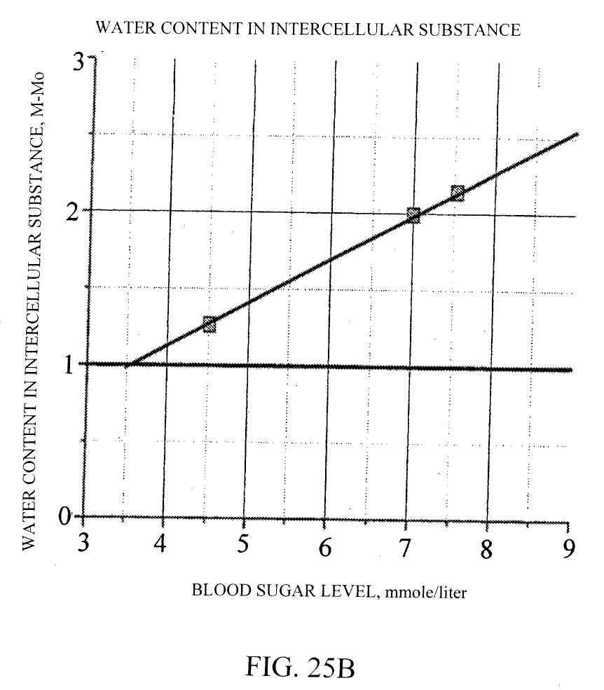

Resulting from the experimental studies carried out using an experimental instrument (FIG. 7) operation principle of which is based on the method mentioned above, unequivocal relationship between blood sugar level and water content in the intercellular substance. Resulting trans-capillary flow, water flow density through the epidermis and tissue heat production have been also established to be unequivocal functions of blood sugar level. FIGS. 9 and 25 present the experimental results that prove lineally proportional relationship between water content in the intercellular substance and blood sugar level. The physical mechanism providing for a linear relationship between water content in the intercellular substance and blood sugar level, is described in the section “Biophysical fundamentals: physics of intercellular substance”. FIG. 5 shows lineally proportional relationship between hydraulic pressure and blood sugar level which has been obtained within the frames of the studied theoretical model. The lineally proportional relationship between water content in the intercellular substance and blood sugar level is a direct sequence of the relationship presented in FIG. 5.

The method allows for a highly accurate measurement of blood sugar level and sugar absorption rate by tissue cells.

Thus, the developed device is in fact a micro calorimeter allowing for determining blood sugar level and sugar absorption rate by a tissue. Measurement accuracy of the method described above is by more than order higher than measurement accuracy of the other methods for monitoring blood sugar level certified by the FDA.

In the section “Examples of practical uses”, experimental results of the comparative measurements of blood sugar levels performed using the experimental instrument (FIG. 7) and using a standardized meter of blood sugar level to conduct control measurements are presented (FIGS. 9 to 20).

Water content in the intercellular substance, capillary pressure, water flow density through the epidermis and resulting trans capillary flow through the epidermis are interrelated with blood sugar level and ambient temperature by the following equations:

J_{ECL}=J_{resulting tcf}=F(C,Cₒ,T,Tₒ)

P_{capillary}−P_{o capillary}=F(C,Cₒ,T,Tₒ)

Pₜᵢₛₛᵤₑ−P_{osmotic}=F(C,CₒT,Tₒ)

m_{ics}−m_{0mcs}=F(C,Cₒ,T,Tₒ)

Here,

C is blood sugar level;

C₀ is blood sugar level wherein tissue pressure is equal to zero.

T is air temperature.

T₀ is air temperature wherein tissue pressure is equal to zero.

A more exact expression for water content in the intercellular substance comprises an additional variable which takes into consideration fluctuations of atmospheric pressure P_{atm.}, and it has the following form:

m_{ics}−m_{0mcs}=F(C,Cₒ,T,Tₒ,P_{atm.})

The expressions for water flow density through the epidermis, resulting trans-capillary flow through the epidermis, tissue pressure and capillary pressure have a similar form.

A functional relationship between pressure in the cardiovascular system and biochemical blood composition the physical mechanisms of which are described in the section “Biophysical fundamentals: physics of intercellular substance”, allows for determining blood sugar level using measurement of practically any of the parameters characterizing the cardiovascular system. To the number of such parameters bong the following parameters: arterial and venous pressure, hydraulic vascular resistance<heart rate and other parameters.

The method of measuring blood sugar level described in the section “A method for measuring level of biochemical blood components by the content thereof in the epidermal corneous layer”, is characterized by that blood sugar level is determined by measuring a time course of sugar content in the epidermal corneous layer.

### A Method for Measuring Hydraulic Pressure in the Microcirculation System

The method for measuring water amount in a tissue described in the section “A method for measuring water amount in the intercellular substance”, allows for determining values of the parameters characterizing state of the intercellular substance in microcirculation of a local tissue site in the regimen of continuous measurement in a real time. In particular, the method allows for determining osmotic pressure value in the intercellular substance and hydraulic pressure in the microcirculation system.

Furthermore, the methods allows for quantitative determining values of the following parameters: the maximum pressure in the microcirculation system (pressure in a capillary arterial end), the minimum pressure in the microcirculation system (pressure in a venous arterial end), osmotic pressure of the intercellular substance, values of trans-capillary flows (resulting, filtration and absorption ones), filtration coefficient of the intercellular substance, water content in the intercellular substance, a value of capillary hydraulic resistance.

The method is based on measuring a parameter characterizing the state of a local tissue site at different values of external pressure to a controlled site surface. Such parameters characterizing the state of a local tissue site are for example: water flow density through the ECL, tissue pressure (osmotic pressure of the intercellular substance), water amount in the intercellular substance.

A method for measuring the parameters of microcirculation and the intercellular substance listed above based on measuring water flow density through the ECL, supposes the following steps:

1) measuring water flow density through a local site of the ECL and ambient air temperature;

2) measuring relationship between water flow density through the ECL and external pressure exerted to a local controllable tissue site;

3) determining microcirculation parameters of a local tissue site by a character and breaks, obtainable according to section 2) of the interrelation.

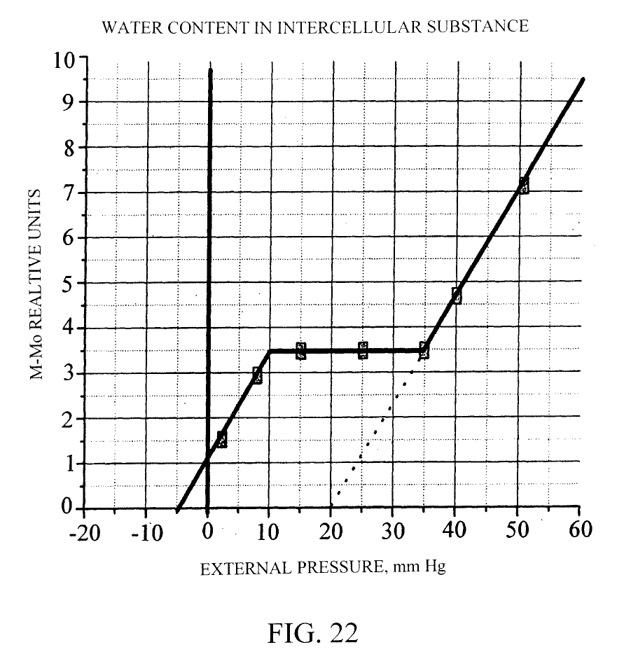

FIG. 22 shows a typical diagram of the relationship between amount of water in the intercellular substance and external pressure value. The values of external pressure wherein typical breaks are found correspond to the minimum and maximum pressure in the microcirculation system. A mean pressure value determined by the maximum and minimum pressure is equal to a mean value of capillary pressure. The slope of linear relationship at the initial and terminal sections allow for determining a filtration coefficient of the intercellular substance for water. The intersection point of the terminal linear section with the axis of pressures corresponds to difference between osmotic pressure of the intercellular substance and blood plasma oncotic pressure.

The possibilities of measuring different microcirculation parameters of a local tissue site, in particular the possibility of measuring water amount in the ECL and the skin intercellular substance as well as the possibility of measuring a tissue filtration coefficient for water allow for using the method in cosmetology to assess efficacy of cosmetic creams as well in dermatology to diagnose pathological skin conditions (un particular, to diagnose and to monitor psoriasis).

### A Method for Measuring Osmotic Pressure of the Intercellular Substance

FIG. 22 shows the relationship between an amount of water in the intercellular substance and external pressure. The intersection point of the initial section of this relationship line with the abscissa axis (the value of external pressure onto a tissue surface in mm Hg) determines a value of an excessive hydraulic pressure (a motive force of volume water flow through the epidermis). The relationship presented in FIG. 22 also allows for determining an absolute value of osmotic pressure of the intercellular substance.

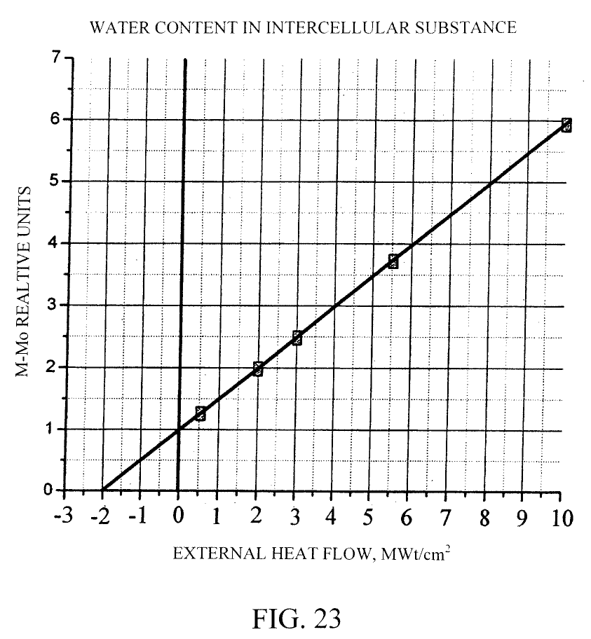

FIG. 23 shows the relationship between an amount of water in the intercellular substance and an external heat flow value. The intersection point of the initial section of this relationship line with the abscissa axis (an external heat flow density directed to the body surface in power units in MWt/cm²) determines an absolute value of water flow density through the ECL or the power of a steam cooling process. The relationship presented in FIG. 23 also allows for determining an absolute value of an excessive water amount M−M₀ (where M₀ is an amount of water in the intercellular substance in a value of osmotic pressure equal to zero) or an amount of water which determines swelling the intercellular substance.

An absolute value of water flow density through the epidermis determinable from the diagram presented in FIG. 23 and an absolute value of a motive force of volume water flow determinable from the diagram presented in FIG. 22, allow for determining a value of the intercellular substance's filtration coefficient war water.

The described method of measurement allows one not only to determine an absolute value of water content in the intercellular substance but it also allows for normalizing this parameter by air temperature and by blood sugar level. The possibility of such normalization allows for determining a deviation from the norm of the measured parameter characterizing a state of the intercellular substance.

The method for measuring an excessive water content (an amount of water in the intercellular substance determining swelling thereof) stipulates the following steps:

1) measuring an amount of water in the intercellular substance using the earlier described methods;

2) measuring the relationship between an amount of water in the intercellular substance and an external heat flow (and/or an external pressure) and determining a value of an excessive water amount (an amount of water determining swelling the intercellular substance);

3) measuring blood sugar level and air temperature;]

4) normalizing the obtained value of water amount in the intercellular substance to the room temperature (20° C.) and to the norm of blood sugar level (5 mM/L).

5) determining a deviation of a water amount in the intercellular substance from the normal amount thereof.

The described method allows for determining changes in state of the intercellular substance by measuring an amount of water in the intercellular substance and comparing the obtained value with the normal value.

### Determination of the Physiological Norm

In the section “Biophysical fundamentals: physics of intercellular substance” synchronization and inter-adjusted functioning the microcirculation and cellular metabolism of a local tissue site was shown to be accomplished due to specific physical characteristics of the intercellular substance.

In the section “Osmotic pressure of the intercellular substance” the method of a practical measurement of the parameters characterizing a physical sate of the intercellular substance have been given consideration. Such parameters, which characterize a state of the intercellular substance, are osmotic pressure and an excessive amount of water determining swelling the intercellular substance.

In practice, measuring an absolute value of an excessive water amount in the intercellular substance, allows for determining a physical sate of the intercellular substance, which determines physiological functioning a local tissue site. Deviation of a physical sate of the intercellular substance from the norm, leads to deviations of the physical sate from the norm.

The physiological norm can be determined in the following way. A functional sate of a local tissue site corresponds to the physiological norm in a case if a physical sate of the intercellular substance corresponds to a state, which is characterized by absence of volume effect or, in other words, if osmotic pressure of the intercellular substance (a tissue pressure) is equal to zero. A tissue pressure equal to zero is achieved at air temperature equal to (about) 20° C. and blood sugar level equal to (about) 5 mM/L. A value of a motive force of water volume flow, the swelling coefficient of the intercellular substance, a water flow density through the epidermis as well as an excessive water amount, which determines swelling the intercellular substance, are under these conditions equal to zero. A resulting trans-capillary water flow is equal to zero and a filtration flow is equal to an absorption flow. A zero level of a tissue pressure corresponds to the atmospheric pressure.

An excessive water amount determining swelling the intercellular substance, and a value of a motive force of a volume flow, are an indicator, which is sensitive to different external effects and diseases. The described method allows for quantitative determining with a high accuracy of a deviation from the norm of a physical state of the intercellular substance of a local tissue site and a s a direct sequence, determining a deviation from the norm of a functional (physiological) state of a controlled local tissue site.

A method for measuring a motive force of a tissue fluid volume flow, osmotic pressure of the intercellular substance and an excessive water amount in the intercellular substance (an amount of water, which determines swelling the intercellular substance) may be used to diagnose different diseases. A method for diagnosing a functional state of a local tissue site based on the method for measuring water content in the intercellular substance, has been given consideration in the section “A method for a functional diagnosis of a local tissue site”.

### A Method for Diagnostics of Cardiovascular Disorders

In the section “Biophysical fundamentals: physics of intercellular substance” the physical characteristics of the intercellular substance and the mechanisms determining an unequivocal relationship between a biochemical composition of the blood, air temperature and hydraulic pressure distribution in the blood circulation system, have been given a detailed consideration.

In particular, a distribution of the intravascular hydraulic pressure in fixed values of external temperature was shown to be unequivocally determined by blood sugar concentration.

In a general case, hydraulic pressure in the blood circulation system is lineally proportionally dependent on blood sugar level and air temperature. In practice, by measuring air temperature and blood sugar concentration, one can unequivocally determine through calculation a hydraulic pressure in different parts of the circulation system.

For example, at blood sugar concentration equal to 4.5 mM/L, pressure distribution in the circulation system is characterized by the following values (in mm Hg): a mean blood pressure is 100, pressure at a capillary arterial end is 54, a mean capillary pressure is 25, pressure at a capillary venous end is 7.

The method allows for determining the following parameters of the cardiovascular system by measuring air temperature and blood sugar level: typical hydraulic pressure values in the circulation system; arterial, venous and capillary hydraulic resistance; values of trans-capillary flows (a resulting, filtration and absorption ones); heart rate and power of cardiac contractions. Under normal conditions at a fixed air temperature, changes in blood sugar level lead to lineally proportional changes in the blood circulation system pressure. The other parameters characterizing a state of the cardiovascular system, are also functions of blood sugar level.

The method for diagnosing cardiovascular disorders stipulates the following steps:

1) measuring air temperature and blood sugar level;

2) determining by calculation a value of a controllable parameter characterizing the cardiovascular system by values of air temperature and blood sugar level using the technique described in the section “Biophysical fundamentals: physics of the intercellular substance”. As such parameter, hydraulic pressure in the circulation system may be for example chosen;

3) determining by measurement a value of a controllable parameter characterizing the cardiovascular system;

4) determining a deviation of a value of a controllable parameter obtained be measurement, from a value thereof determined by calculation by measurements of blood sugar level and air temperature and determining a character and a reason of deviation of the parameter from the norm.

The technique allows for determining parameters of the cardiovascular system by the known values of temperature and blood sugar level. The following ones belong to a number of such parameters: a mean capillary pressure; pressure at venous and arterial capillary end; arterial, venous and capillary hydraulic resistance; a resulting trans-capillary flow.

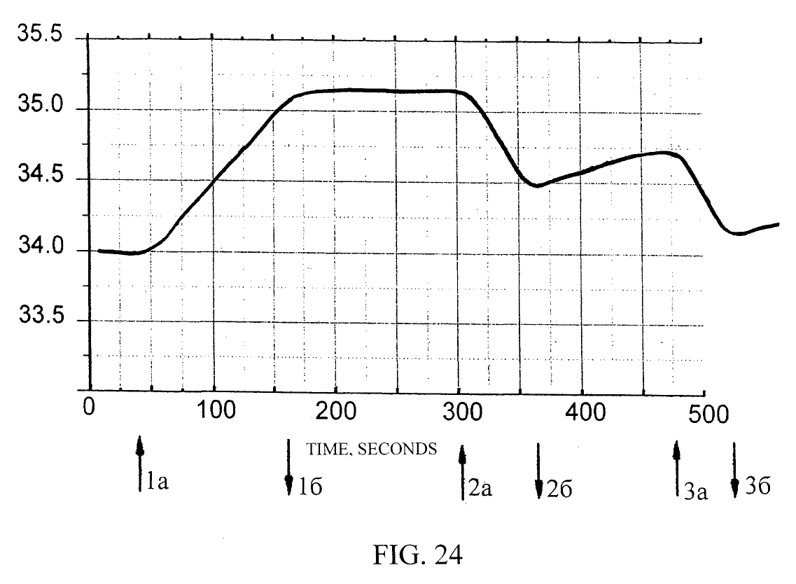

A deviation of the parameters' values obtained by direct measurement from these parameters determined by measuring temperature and blood sugar level (“the norm”) is a direct indication to pathological disorders in the cardiovascular system. In particular, the described method for diagnosis allows for diagnosing pathological conditions of the cardiovascular system, which are characterized by elevated blood pressure (hypertension) and conditions, which are characterized by a lowered blood pressure (hypotension). The diagrams presented in FIG. 24 as well as in FIGS. 1-5 clarify the method of diagnosis described above.

FIG. 24 shows the diagrams of osmotic pressure of the intercellular substance and intra-capillary hydraulic pressure depending on the dimensionless parameter “α” around the point corresponding to a value of an input pre-capillary pressure. Change in the intercellular substance's properties as a result of different disorders, leads to typical deviations of osmotic pressure equilibrium distribution form the kind shown in FIG. 1 and FIG. 24 (the diagram is “the norm”). Resulting from such deviations, mechanical equilibrium in the system “the intercellular substance—a capillary vessel” is achieved at higher (the diagram “elevated pressure”) or lower (the diagram “lowered pressure”) values of intra-capillary hydraulic pressure. Thus, deviations of a pressure value in the cardiovascular system from the pressure, which is determined by calculation originating from the values of blood sugar level and temperature allows for diagnosing disorders of the cardiovascular system, in particular, determining states with elevated and lowered pressure.

### A Method for Diagnostics of Cardiovascular Disorders: Monitoring Condition of the Cardiovascular System in Patient with Diabetes

The method for diagnostics described in the previous section “A method for diagnostics of cardiovascular disorders” allows for performing diagnostic monitoring of the blood circulation system's condition in patients with diabetes. Diabetic condition is known to be accompanied by disorders of the cardiovascular system. In diabetes, the both peripheral and central blood circulation systems are known to be subjected to pathological changes.

Elevated blood sugar level is a cause of pathological changes occurring in the blood circulation system. Elevated blood sugar level leads to elevated values of pressure in the blood circulation system. The biophysical mechanism determining an unequivocal relationship between pressure in the microcirculation system and blood sugar level has been given a detailed consideration in the section “Biophysical fundamentals: physics of the intercellular substance”. A prolonged maintenance of an elevated pressure exceeding the norm in the blood circulation system is accompanied by an increased load on cardiac and vascular work and as a sequence, it leads to the development of pathological cardiovascular disorders.

For the indicated reason, monitoring the condition of circulation in diabetic patients is by now an actual and burning task. Such monitoring will allow patients with diabetes to timely correct therapy and to avoid the development of chronic cardiovascular diseases, which are currently the main cause of lethal outcomes in patients with diabetes. In particular, the described method allows for early diagnosis and monitoring the disease known as “a diabetic foot”.

### A Method for Diagnostics of a Functional (Physiological) State of a Local Live Tissue Site

In the section “Biophysical fundamentals: physics of the intercellular substance” distribution of hydraulic pressure in the microcirculation system as well as distribution of osmotic pressure of the intercellular substance in a tissue volume between blood capillaries are shown to be determined by a physical (phase) state of the intercellular substance. On the other hand, a physical state of the intercellular substance is an unequivocal function of biochemical blood composition, air temperature and intra-capillary hydraulic pressure. Synchronization of volume flows of a substance and heat (including blood circulation in the system of blood capillaries, tissue liquid circulation in the intercellular substance and circulation of sugars and cellular metabolism products) is effected due to specific physical characteristics of the intercellular substance. Intensity of a substance and heat flows such as flows of a tissue liquid, glucose and other dissolved substances and heat transfer flow to the body surface are equivocal functions of a phase state of the intercellular substance.

Change in physical characteristics of the intercellular substance of a tissue local site resulting from the development of pathological disorders of different nature, leads to disorders and deviations of a mutually adjusted (synchronous) functioning of the system: a blood capillary—the intercellular substance—a tissue cell.

The method for measuring the parameters characterizing a physical state of the intercellular substance described in the section “A method for measuring osmotic pressure of the intercellular substance” opens principally new possibilities for diagnosing a functional (physiological) state of a local live tissue site.

The method for diagnostics stipulates the following steps:

1) measuring a value of a parameter characterizing a state of the intercellular substance, for example, water amount in the intercellular substance, osmotic pressure or a resulting trans-capillary flow;

2) measuring air temperature and blood sugar level;

3) determining a calculated value of a parameter characterizing a state of the intercellular substance;

4) determining a deviation of a value of a parameter obtained be measurement, from a value thereof determined by calculation by measurements of air temperature and blood sugar level;

5) determining by the deviation value (section 4) a character of the deviation and a degree of a pathological state of a local site intercellular substance.

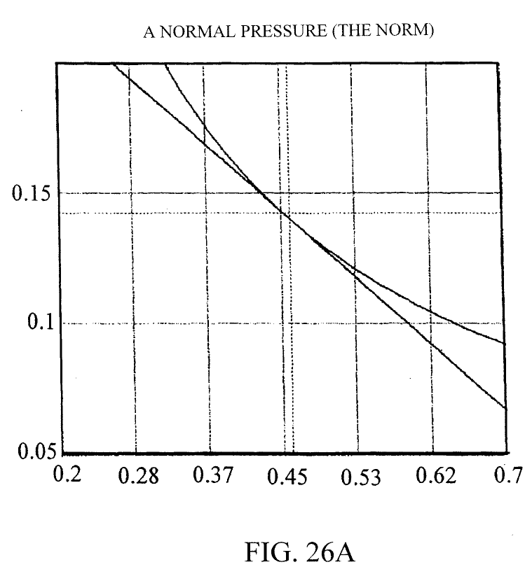

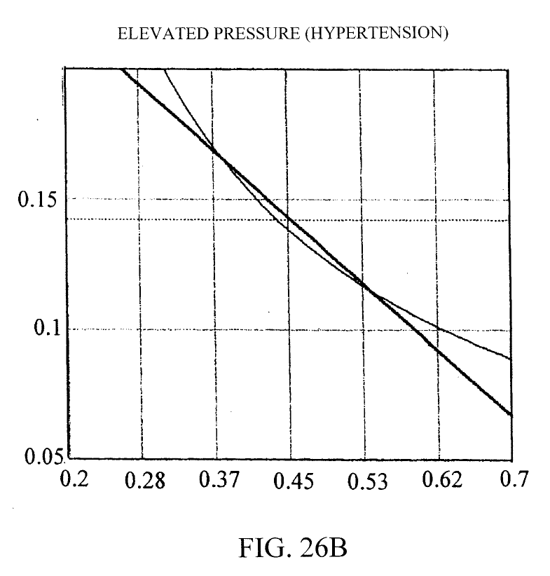

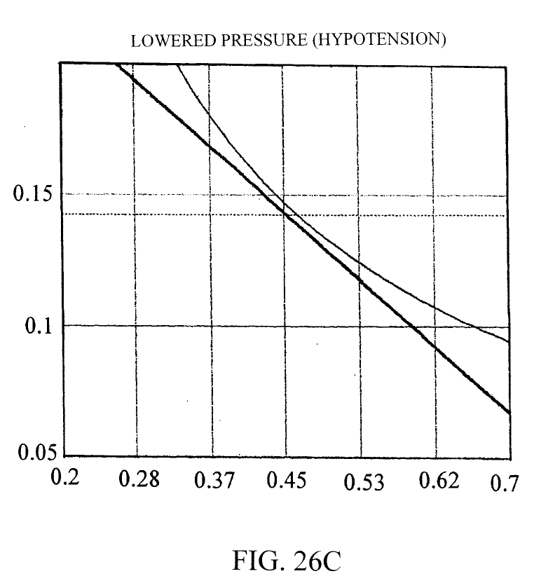

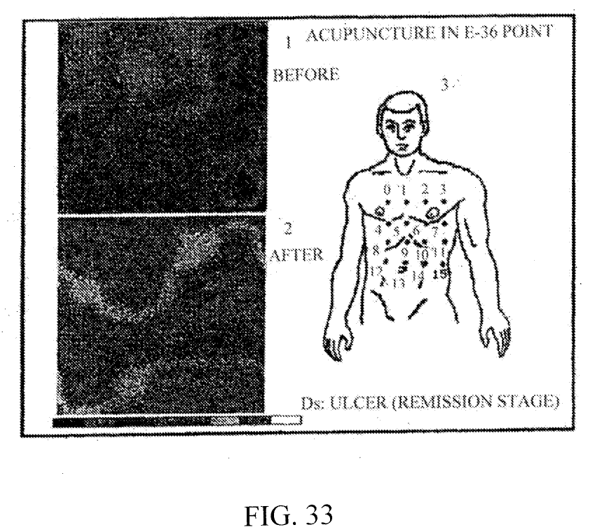

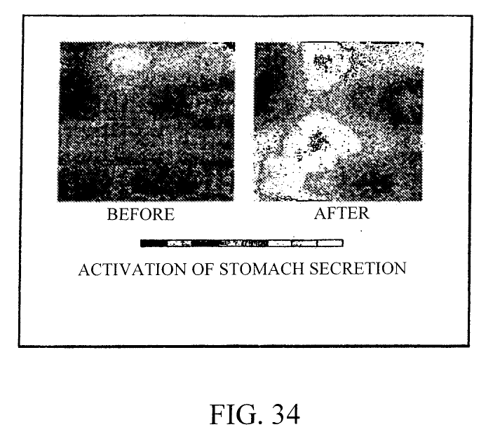

Another method for diagnosing a functional sate of a local tissue level is based on an on-line recording a dynamic reaction of a parameter characterizing a state of the intercellular substance in response to a weak external effect. Hereinafter, under a dynamic reaction a time course of change in a parameter characterizing a tissue state in response to an external effect is meant. Effects of different nature (physical, physiological or chemical) belong to the effect leading to change in a state of the intercellular substance. For example, external heat flow, external pressure etc. belong to external physical effects. The typical examples of dynamic reactions caused by change in water amount in the intercellular space resulting from the effects of different nature are presented in FIGS. 22, 23, 26, 32, 33.

By changing external temperature or heating (cooling) the body surface, one can change swelling degree of the intercellular substance or water amount in the intercellular space. Similar effect can be achieved due to change in external pressure relative atmospheric pressure. A local decompression (vacuum) causes compression of the intercellular substance and an excessive pressure, on the contrary, leads to swelling thereof. In FIGS. 22, 23, 26, 32 the experimental results on studying the effects of the factors mentioned above on a local tissue site.

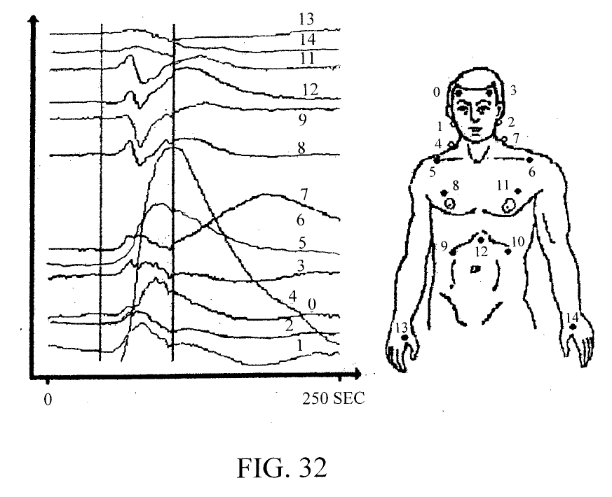

The effects described above are a sequence of physical characteristics of the intercellular substance. For this reason, by value and character of a dynamic reaction of the parameter characterizing a state of the intercellular substance, one can determine possible deviations of the intercellular substance properties from the norm and to diagnose a physiological state of a tissue local site. For example, a local thermal effect of electromagnetic radiation (infrared or optic) on the body surface leads in a real time to a typical local reaction of the parameters characterizing a state of the intercellular substance of a local controllable site. In such effect, osmotic pressure of the intercellular substance changes that results in rise in hydraulic pressure in the microcirculation system and as a sequence, elevation of a resulting trans-capillary flow and water flow density through a local site of the ECL occurs. A typical specificity of a reaction corresponding to a physiological norm in response to an external thermal effect is that change in steam cooling power determinable by change in water flow density through the ECL, appears to be exactly equal to a heat effect power. A thermal effect with the power 1 MWt/cm² leads to increase in a resulting trans-capillary flow value and water flow density through the ECL (determining intensity of a steam cooling process), which increase is equivalent to rise in steam cooling intensity by 1 MWt/cm². A typical time constant of forming such reaction is several seconds. Change in the intercellular substance properties occurring as a result of disorders and pathologies of different nature, leads to change in a typical reaction in response to a weak effect of a physical nature. The typical experimental results on studying the effect of heat flows on a state of the intercellular substance are presented in FIGS. 22 and 32.

The method for diagnostics supposes the following steps:

1) a real-time measuring a value of the parameter characterizing a state of the intercellular substance (for example, water amount in the intercellular substance);

2) a local dosed effect on a tissue using physical factors of a weak intensity (examples of physical factors: an external thermal effect, external pressure, a direct electric current and a constant magnetic field);

3) a real-time measurement of a dynamic reaction of a recorded parameter in response to an external effect (for example, a heat flow) and determining water flow density value through the epidermis;

4) determining a physiological state deviation of a local tissue site from the norm and diagnosing a functional state by water flow density value through the epidermis and by a dynamic reaction character (intensity of reaction, time delay, time course character).

Another possibility of a functional diagnosis of a local tissue site is described in the section “A method for measuring osmotic pressure of the intercellular substance” and it is based on measuring relationship between water amount in the intercellular substance and an external effect.

5) Measuring water amount in the intercellular substance depending on external thermal effect (FIG. 23) allows for determining the amount of water, which determines swelling the intercellular substance. The described method allows for not only determining water amount in the intercellular substance, but also normalizing this parameter by air temperature and blood sugar level. The possibility of such normalization allows for determining deviation from the norm of the measurable parameter characterizing the state of the intercellular substance.

In a similar way, the intercellular substance state is diagnosed using effects (physical and physiological) of a different nature. To the number of such physical effects also relate an external pressure, a local decompression, a direct electric current, a constant magnetic field and others. Examples of physiological effects are a sugar test and different medicaments exerting effect on the intercellular substance characteristics.

The method for measuring water amount in the intercellular substance determining swelling the intercellular substance, supposes the following steps:

6) measuring water amount in the intercellular substance using the methods described above;

7) measuring relationship between water amount in the intercellular substance and an external heat flow (or an external pressure) and measuring water amount determining swelling the intercellular substance;

8) measuring blood sugar level and air temperature;

9) normalizing the obtained water amount value in the intercellular substance to a room temperature (20° C.) and the normal blood sugar level (5 mM/L);

10) determining a deviation of water amount value in the intercellular substance from the normal amount thereof.

The described method allows for determining changes in the intercellular substance state by measuring water amount in the intercellular substance and comparing the obtained value with the normal values.

The method for measuring an excessive water amount (or the water amount determining swelling the intercellular substance) admits a simple qualitative determination of the physiological state of a local tissue site through the notion of the intercellular substance physical sate.

Determination of the physiological norm is given consideration in the section “Determining the physiological norm”.

A functional sate of a local tissue site corresponds to the physiological norm in the case if the intercellular substance physical sate corresponds to the state which is characterized by lacking volume effects or, in other words, is osmotic pressure of the intercellular substance (tissue respiration) is equal to zero. Tissue respiration equal to zero is achieved at air temperature equal to 20° C. and blood sugar level equal to 5 mM/L. The value of a motive force of a volume water flow, the swelling coefficient of the intercellular substance as well as an excessive water amount determining swelling the intercellular substance are under these conditions equal to zero.

The excessive water amount determining swelling the intercellular substance and the value of the volume flow moving force ate indicator that is sensitive to different external effects and diseases. The described method allows for quantitatively determining deviations from the norm with a high accuracy of the sate of the intercellular substance of a local tissue site.

The methods of diagnostics described above, may be used for early diagnosing different diseases the development of which is accompanied by a change in the intercellular substance characteristics. The following diseases relate to such diseases:

malignant tumors the development of which is accompanied by the typical changes in localized tissue sites;

the disease known as “an orange skin” and the development of which is accompanied by the typical changes in the skin and the subcutaneous cellular tissue:

different stages of obesity;

type 1 and 2 diabetes accompanied by the typical changes in the intercellular substance characteristics (for example, tissue sensitivity to insulin) and microcirculation;

some cardiovascular diseases the development of which is accompanied by the typical changes in the intercellular substance and many other diseases.

Furthermore, the described method of diagnosing pathological states of the intercellular substance may be used in cosmetology and esthetic medicine to assess a functional state of the skin as well as to visualize and to assess the effect on the skin of different cosmetic creams and medicaments

To embody “The method for diagnosing a functional (physiological) state of a local tissue site” described in the present section, a device for measuring water amount in the intercellular substance is used. The for measuring water amount in the intercellular substance the accuracy of which exceeds 1%, is described in the section “A method for measuring water amount in the intercellular substance”. This method can be individually used in practice for example, to measure a local humid content in the skin tissue to assess the effect of cosmetic creams.

### A Method for Determining a Tissue Sensitivity to Insulin

### Diagnosis in Diabetic State

The method for measuring blood sugar level described in the section “A method for measuring local tissue metabolism rate” allows for determining blood sugar level by measuring water amount in the intercellular substance of a local tissue site and air temperature. The physical mechanisms determining relationship between the intercellular substance properties and sugar concentration are described in the section “Biophysical fundamentals: physics of intercellular substance”.

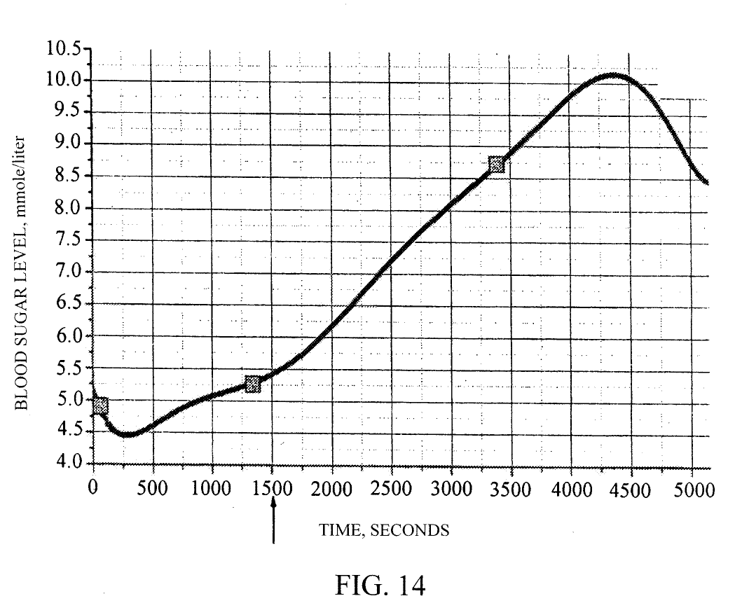

The given method allows for conducting recording blood sugar level in a continuous monitoring regimen (one measurement every 5 to 10 seconds). FIG. 14 shows the results of the continuous monitoring blood sugar level under the conditions of conducting the standard glucose tolerance test (“a continuous sugar curve”). For comparison, the modern manuals determine as “a sugar curve” several measurements (as a rule, 3 to 4) performed with blood samples drawn from hand fingers with an interval between measurements of about 30 minutes. The experimental results presented in FIG. 14, were obtained using the experimental instrument the view of which is presented in FIG. 6. The operation principle of the experimental instrument is described in the section “The electrometric method for measuring water amount in the intercellular substance”.

The method for recording a sugar curve based on a continuous measuring a temporal dynamics of a local parameter characterizing the intercellular substance state of a local site, opens principally novel opportunities for diagnosing pre-diabetic state and determining sensitivity of a local tissue to insulin.

Disorder of glucose tolerance. Modern manuals on medicine determine disorder of glucose tolerance (DGT) as blood glucose concentration during the oral glucose tolerance test lying in the interval between normal and diabetic values (2 hours after administering 75 g glucose—from 7.8 to 11.0 mM/L). DGT may probably be given consideration as to a pre-diabetic state, while not all subjects with DGT develop diabetes. In USA every tenth adult individual has DGT the rate thereof increasing with age achieving every fourth among persons aged from 65 to 74 years. The epidemiological studies carried out in different countries indicate to a close relation between DGT and obesity. For example, the study carried out in the USA, has found that a mean EBW (excessive body weight) in persons, who consequently developed DGT, was significantly higher than in individuals with a normal EBW. The study carried out in Israel has established that a history of a high EBW was accompanied by a raised frequency of DGT development over a 10-year period.

The method for recording a sugar curve described above, allows for determining DGT in a continuous monitoring regimen with a higher accuracy. In particular, the method is efficient for determining type 2 pre-diabetic state.

### A Method for Determining a Tissue Sensitivity to Insulin

The method for a continuous recording a time course of a local tissue metabolism (sugar absorption rate by a local tissue site) described in the section “A method for measuring a local metabolism rate” allows for determining a tissue sensitivity to insulin by a time course of sugar absorption rate by a tissue. The method for determining tissue sensitivity to insulin is based on a continuous recording a time course of sugar absorption rate by a tissue. Water amount in the intercellular substance of a local tissue site is measured and changes in temporal dynamics resulting from external effects leading to the typical changes in tissue sensitivity to insulin are recorded. Effect on a tissue of some external physiological and physical factors is known to lead to reversible changes in tissue sensitivity to insulin. To the number of such factors belong in particular muscular load and temperature effects [2]. To external effects, which cause reversible changes in tissue sensitivity to insulin, belong the effect which lead to reversible changes in a phase state of the intercellular substance. The external physical parameters, which determine a phase state of the intercellular substance, have been given consideration in the section “Biophysical fundamentals: physics of intercellular substance”. To the number of such external physical factors belong the following: an external pressure; a local decompression; an external temperature; electromagnetic radiation causing volume heating of a tissue; a weak direct electric current; a constant magnetic field; a local muscular load to a tissue and others.

The method for determining tissue sensitivity to insulin supposes the following steps:

1) measuring a local tissue metabolism (sugar absorption rate by a tissue) in a continuous monitoring regimen during a standard sugar load (oral administration of 75 g glucose) by measuring water amount in the intercellular substance and air temperature;

2) exerting external physical effect that causes a reversible change in tissue sensitivity to insulin on a controllable local tissue site;

3) determining tissue sensitivity to insulin by the character of a local metabolism time course.

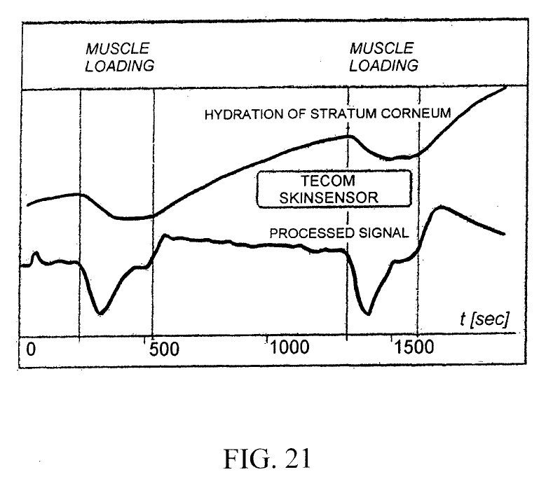

An example of a practical embodiment of the method is presented in FIG. 21.

In the presented experiment (FIG. 21), a time course of water content in the intercellular substance caused by muscular load is recorded in a real time. Muscular load leads to typical changes in time course: reduction in the recorded parameter occurs and growth thereof after a typical time interval equal to 1 to 2 minutes begins. Such character of water content changes in the intercellular substance is associated with the typical blood sugar level changes under the muscular load conditions. Reduction in the intercellular substance water content after beginning the load, is caused by reduction in local blood sugar and the intercellular fluid content. Dropping sugar the intercellular fluid sugar level at the initial section of the temporal dynamics line is associated with rise in a local tissue sensitivity in response to muscular load. A subsequent rise in the intercellular substance water content leading to increase in water content in the ECL is caused by sugar content rise in the tissue fluid resulting from glycogen cleavage comprised in muscular cells.

### A Method for Managing a Tissue Fluid Transport and Lymph Drainage

In the sections “Biophysical fundamentals: physics of intercellular substance” and “Biophysical fundamentals: microcirculation mechanisms of tissue fluids” physical properties of the intercellular substance as well as the physical mechanism providing for blood circulation in the capillary system and tissue fluid transport in the intercellular space have been given consideration. In particular, in these sections osmotic pressure of the intercellular substance, elastic pressure (elastic strain of the intercellular substance) and hydraulic pressure in the microcirculation system were shown to be unequivocally determined by the parameters which are variables of the intercellular substance state. The variables of the intercellular substance state are an external pressure, temperature and plasma glucose concentration.

The method for managing a tissue fluid transport is based on the possibility of changing a volume flow of the tissue fluid circulating in the intercellular space by affecting the intercellular substance with weak effects of physical and chemical nature. External pressure, heat flow, a constant magnetic field, direct electric current and others relate to the external physical effects using which managing the tissue liquid transport is possible.

In FIGS. 22, 23, 24, 25, the experimental study results of the effects of different physical factors on a local tissue site are presented. The experimental results presented in these figures prove the possibility of changing a local water content in the intercellular substance using physical effects of a weak intensity and they thereby prove the possibility of efficient managing the tissue fluid transport using external physical and chemical effects.

By changing external pressure (FIG. 22), one can change swelling degree of the intercellular substance (water content in the intercellular substance) and, as a sequence, the tissue fluid volume flow in the intercellular substance and in the capillary vascular system. An excessive external pressure on a local body surface leads to swelling the intercellular substance and a local decompression (vacuum), on the contrary, results in compression of the intercellular substance. In such method of compressing the intercellular substance, there occur increase in capillary vascular lumen and increase in the lumen of the channels through which the tissue fluid circulates. Such local effect results in a raised volume flow rate through the capillary vessels and a volume flow of the tissue fluid circulating in the intercellular substance.

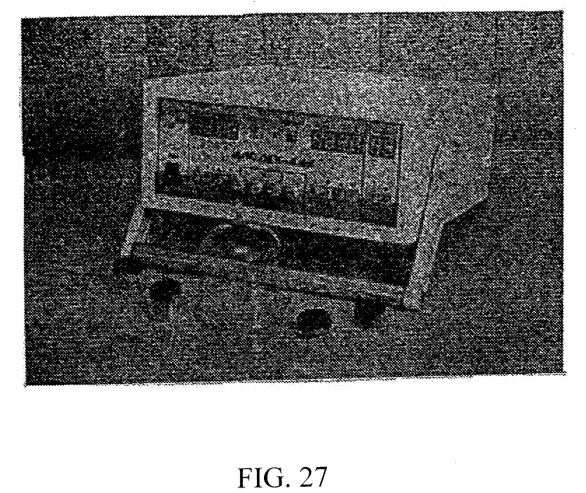

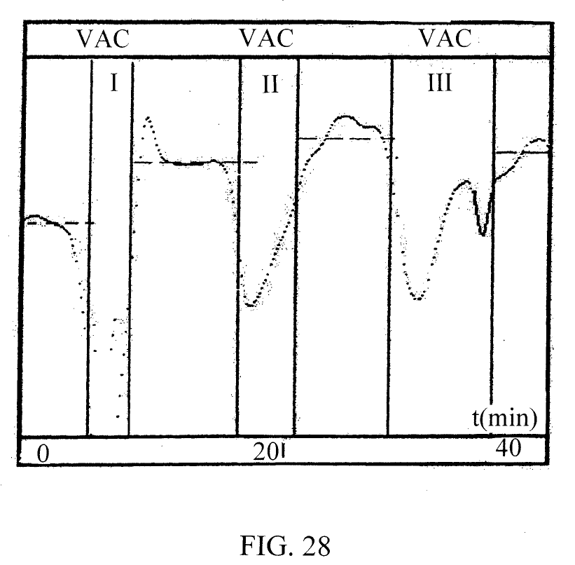

FIG. 28 presents the experimental results of studying the effect of a local decompression on the intercellular substance state. A local pressure lowering relative to atmospheric pressure is seen to lead to the effect of a diminished water content in the intercellular substance caused by the effect of the intercellular substance compression effect. A local decompression in these experiments was effected using the local decompression instrument Alodec—4ak the appearance of which is shown in FIG. 27. The body surface is locally affected using a special vacuum applicator (a specific “cup”) inside which a dosed decompression regimen is maintained.

Such method of a local pulsing effect on a tissue results in periodic pulsations of osmotic and elastic pressure of the intercellular substance as well as hydraulic pressure in the capillary vascular system in a tissue volume under the vacuum applicator. Such effect leads to volume pulsations of the intercellular substance characterized by the occurrence of pulsating liquid flows circulating in the system “the blood circulation capillaries—the intercellular space—the lymphatic drainage system”. Such method using an external effect provides for managing a tissue liquid transport and lymphatic drainage of a local tissue site.

A physiotherapeutic effect of such exposure becomes clear if one takes into consideration that a volume flow of the tissue fluid provides for delivery of nutrients and oxygen to tissue cells and draining products of cellular metabolism into the blood circulation system and the lymphatic system. This process initiated by an external effect results in beginning an efficient supply of a tissue with sugars, nutrients and oxygen. As a natural sequence, the processes of cellular metabolism and general metabolism are accelerated: metabolism rate of tissue cells is growing that is a stimulating growth factor of cells and regeneration of tissues.

A smooth regulation of a vacuum degree in the applicator allows for regulating and establishing a tissue layer depth wherein the drainage effect stimulated by an external effect is caused. The drainage effect “X” is interrelated with the negative pressure “P” by the following equation:

P=F(P₀,X,L₀)

where P₀ is a tissue pressure

L₀ is a thickness (depth) of a tissue volume under the applicator

A value of a tissue pressure P₀ can be determined by measuring water amount in the intercellular substance or blood pressure. A thickness (depth) of a tissue volume under the applicator can be determined by measuring a circle perimeter of the controlled body site.

A smooth regulation of the rate and porosity of pneumo pulses allows for regulation and establishment of a volume flow value of tissue fluid and lymph drainage.

A similar effect is achievable by change in external temperature or cooling (heating). A local cooling of the body surface causes contraction of the intercellular substance and heating a tissue leads to swelling thereof. FIGS. 23 and 24 present the experimental results on studying the effect of external heat flows on the intercellular substance state. A local effect of heat flow on the body surface is seen to result in increased water content in the intercellular substance of a local site caused by swelling the intercellular substance. On the contrary, a local cooling the body surface reads to diminishing water content in the intercellular substance resulting from contraction of the intercellular substance.

The effects of contraction and swelling a tissue can be stimulated also using a weak direct electric current and a constant magnetic field. A mechanical equilibrium of the system “the intercellular substance—a capillary” which determines water content in the intercellular substance proved to be also sensitive to weak constant electric and magnetic fields. The mechanism of such sensitivity becomes clear if one takes into consideration that direct electric current leads to a change in an equilibrium distribution of electric ions of tissue fluid in a tissue volume that in its turn results in disorder of the system of mechanical equilibrium and in change in water content in the intercellular space. Electric current directed from inside toward the skin surface results in the effect of swelling the intercellular substance. On the contrary, change in direction of electric current results in a contraction effect of the intercellular substance.

The mechanism of sensitivity to a constant magnetic field is based on the fact that transfer of charged ions in a tissue volume is effected by flows of intercellular fluid and a constant magnetic field leads to redistribution of these flows and to disorder of the system's mechanical equilibrium.

Thus, the method for managing a tissue fluid transport and lymph drainage is based on the effect on a tissue using different physical factors, which cause reversible changes in water content in the intercellular space. To the number of the physical factors using which managing a tissue fluid transport is possible relate the following: a local superficial cooling (heating) or a thermal electromagnetic radiation; local decompression and excessive pressure; direct electric current and a constant magnetic field; acoustic fluctuations (a law frequency vibration, ultrasound etc.) and other factors.

Local effects of a low intensity as a rule lead to the effects described above. Typical powers and values of physical effects are as follows: electromagnetic radiations 0-20 MWt/cm²; local decompression values 0-100 mm Hg; direct electric current values 0-100 nA; values of a constant magnetic field intensity 0-50 MT.

The method for managing a tissue fluid transport described above may be used in treating different diseases. Different diseases may lead to different typical changes in the intercellular substance state.

Diseases accompanied by swelling the intercellular substance state exceeding the norm (the “tissue edema” state) may be treated and prevented using the effects which cause a local contraction of the intercellular substance (a local decompression, cooling).

Diseases accompanied by a lowered water content in the intercellular substance may be treated and prevented using the effects which cause a local a local increase in swelling degree of the intercellular substance (a local compression, heating).

The method for managing a tissue fluid transport stipulates the following steps:

1) measuring water content in the intercellular substance of a local tissue site;

2) determining the intercellular substance state by water content in the intercellular substance;

3) determining a method of external effect and regimen of the effect by the state of the intercellular substance;

4) external effecting;

5) controlling efficacy of exposure by measuring water content in the intercellular substance.

To the number of such diseases which can be efficiently treated using the instant method relate the following:

vertebral diseases, in particular osteochondrosis;

sexual disorders, in particular erectile dysfunction; articular diseases;

the disease known as “the orange skin disease” and other diseases;

diseases of internal organs.

The method allows for stimulating cellular growth of the breast tissue, it leads to increase in elasticity of the facial tissue and other body parts.

The method for managing a tissue fluid transport given consideration above is also applicable for treating and preventing type 2 diabetes.

### A Method for Diagnosing a Pathological State of Internal Organs

The method for diagnosing consists in a real-time recording spatial-temporal distribution of a parameter characterizing the intercellular substance state of a local superficial site. The parameters characterizing the intercellular substance state of a local superficial site are for example osmotic pressure of the intercellular substance, water content in the intercellular substance, a value of a resulting trans-capillary water flow.

A spatial-temporal distribution is recorded using a multi-channel system the sensors of which are positioned on the controllable body site surface or using a scanning system. FIG. 28 schematically clarifies the method of recording spatial-temporal distribution of a parameter characterizing the intercellular substance state (a dynamic mapping). The typical examples of the spatial-temporal distribution of a local metabolism rate obtained using the multi-channel system (a matrix of sensors 4×4) are presented in FIGS. 28-32.

The possibility of diagnosing the state of internal organs by measuring water content in the intercellular substance of the body superficial layer is based on the intercellular substance characteristics and peculiarities of a non-diffuse heat transfer mechanism from depth to surface. The intercellular substance characteristics and the heat transfer mechanism have been given consideration in the sections “Biophysical fundamentals: physics of intercellular substance”, “Biophysical fundamentals: the mechanism of tissue fluid transport in intercellular space”, “Biophysical fundamentals: the mechanism of a non-diffusion heat transfer from depth to surface”.

Under normal physiologic conditions, temperature of an internal organ (37° C.) is as a rule higher than temperature of superficial tissues (30° C.). Such temperature difference leads to difference in osmotic pressure values of the intercellular substance and hydraulic pressure in the intercellular substance “channels” by which tissue fluid is transported. Tissue fluid is transported from depth to surface resulting from difference of hydraulic pressure. This process provides for heat transfer generated as a result of cellular metabolism from depth to surface and simultaneously maintains a steam cooling process (a non-perceived perspiration).

The development of an internal organ pathological state is accompanied by change in the intercellular substance state of this organ. For example in case when a chronic diseases of an internal organ is characterized by a lowered level of organ metabolism, osmotic pressure of the intercellular substance and pressure in the microcirculation system are also lowered. Tissue fluid circulation rate toward the surface is accordingly lowered. Eventually, this process results in the appearance a spatial non-uniformity of water content in the intercellular substance and rate and density of water flow through the ECL.

Thus, spatial-temporal mapping of water content in the intercellulae substance allows for diagnosing pathological state of internal organs and determining a deviation of organic metabolism from the norm.

The method for diagnosing stipulates the following steps:

1) recording spatial-temporal distribution of water content in the intercellular substance;

2) localizing a problem site by a character of spatial-temporal distribution non-uniformity;

3) determining a differential drop value by measurements of water content in the intercellular substance in the following two points (sites, zones) of the body surface: one directly coinciding with the spatial non-uniformity region and another outside this region;

4) diagnosing by the differential drop in a controllable parameter value in the two surface points.

A method for diagnosing may be also based on comparing values of the parameters obtained by direct measurements with their values obtained originating from blood sugar level measurements and air temperature. Such diagnosing stipulates the following additional steps:

5) measuring air temperature and blood sugar level;

6(determining a calculated value of the parameter characterizing the intercellular substance state;

7) determining a deviation of the parameter value obtained by measurements form the value of this parameter obtained by calculation (by the values of air temperature and blood sugar level);

8) determining character and degree of an internal organ's pathological state by the deviation value (section 7) of the controlled parameter.

The method of measurement described in the section “A method for determining osmotic pressure of the intercellular substance in the microcirculation system” allows for practical realization of “The method for diagnosing a pathological state of internal organs” described above, by the different method. Such method stipulates the following steps:

1) real-time recording a spatial-temporal distribution of water content in the intercellular substance;

2) localizing a problem site by a character of the spatial-temporal distribution and characteristics of water content in the intercellular substance over time;

3) measuring air temperature and blood sugar level;

4) determining by calculation using the measured values of temperature and blood sugar level, values of the microcirculation parameters and the intercellular substance;

5) measuring the parameters characterizing a state of a local tissue site using the method described in the section “A method for determining osmotic pressure of the intercellular substance in the microcirculation system”.

6) diagnosing a state of an internal organ by deviations of values of the parameters obtained by measurements from the values of these parameters obtained by calculation.

Diagnosis using physiological tests and external effects is a variant of the method for diagnosing given consideration above. The method for diagnosing using external effects and physiological loads essentially do not differ from the method described in the section “A method for diagnosing a pathological state of the intercellular substance”.

Physiological tests may be local and general. To the number of physiological tests relate thermal effect, external pressure, local decompression, electric current, local muscular load. An example of a general physiological load is for example a standard sugar load used in performing a glucose tolerance test.

Under the conditions of the mentioned physiological effect the typical reaction of a local metabolism of a superficial tissue site will as a rule be non-uniform in disorders of organ metabolism. A physiological load allows for visualizing internal body regions which are characterized by a disordered tissue metabolism.

FIG. 32 shows the results of a practical use of the method for diagnosing internal organs using spatial-temporal mapping water content in the intercellular substance.

The methods for diagnosing described above, allow for diagnosing a pathological state of internal organs as well as diagnosing diseases the development of which is accompanied by formation of local regions with modified tissue characteristics. To the number of such diseases relate malignant masses or cancer tumors. In particular, the method allows for detecting breast cancer at early stages of development thereof practically at any depth.

### A Method for Diagnosing Breast Cancer

The process of formation and growth of breast cancer is known to be accompanied by typical physiological changes in tissue in the tumor location region as well as by changes in tissue in a superficial region determined by projection of the tumor region to the surface.

The following typical changes can relate to the number of physiological changes occurring in the region of cancer tumor localization:

Elevated level of glucose metabolism characterized by a raised rate of sugar absorption by cancer tissue recorded using a positron-emission tomography;

a high multiplication rate of cancer cells which is not typical for a normal tissue;

a typical tissue condensation recorded by X-ray methods;

typical changes in microcirculation recorded by optic methods.

Typical physiological changes occur also in superficial tissues localization of which is determined by a tumor region projection to the surface. To the number of such changes relate changes in microcirculation characterized by changes in surface temperature recorded using thermo-vision methods.

As cancer tumor grows, a gradual involvement of the surface tissue located over the tumor region inside the breast occurs.

Malignant tumors have an elevated level of glucose metabolism and enhanced tissue sugar consumption and as a sequence, elevated level of heat production.

Among the known methods of diagnosing breast cancer a “gold standard” is an X-ray mammography which allows for detecting and determining localization of a cancer tumor with a high probability. However, the radiographic method does not allow for identification a cancer tumor and distinguishing a cancer tumor from a malignant tumor. In clinical practice, for theses purposes a biopsy method is used which is expensive and painful.

Positron-emission tomography is a method, which allows for detection and identification of malignant neoplasms.

Regions of cancer tissue which are characterized by an increased sugar absorption rate, are detected with a high spatial resolution using a positron-emission tomograph (PET). However, a practical use of the PET for early diagnostics and screening breast cancer is limited, since the equipment is expensive.

Analysis and judging characteristic physiological changes occurring during the development of a cancer tumor which were carried bout based on comprehension of physical properties of the intercellular substance given consideration in the section “Biophysical fundamentals: physics of intercellular substance”, allow for explaining the mechanism of the main changes occurring in the breast tissue affected by cancer.

In the breast tissue affected by cancer a local lowering tissue pressure and contraction of the intercellular substance in the tumor region occur. This process leads to a gradual tissue condensation in the tumor region. Contraction of the intercellular substance leads to increase in the lumen of capillary vessels and channels in the intercellular space along which tissue fluid circulates in the intercellular space and increase in volume flow of tissue fluid. As a result, increase in delivery rate of sugars to a cancer cell occurs. Sugar absorption by the cell and metabolism rate in a local tissue region increase. Such changes probably maintain multiplication process of cancer cells.

Typical changes in tissue also occur in the tissue volume located between the tumor region and projection thereof to the surface. Lowering osmotic pressure of the intercellular substance in the tumor region results in lowering (or leveling) osmotic pressure gradient of the intercellular substance in a direction from the tumor toward the surface. As a sequence, water transport through the epidermis and water content in the intercellular substance of superficial layers, in particular the skin and the ECL, significantly diminish. Reduced intensity of steam cooling with concurrent rise in glucose metabolism rate and heat production, leads to the tissue temperature elevation in the tumor region as well as to rise in a superficial region temperature determined by projection of the tumor region to the surface. Development and growth of the tumor is accompanied by a gradual contraction of the intercellular substance in the region between the tumor and projection thereof to the surface. This process leads to elastic strain occurrence in the direction from the body surface toward the tumor region, which results in a gradual inward traction of the tumor as it grows.

The methods of measurement described above in the sections “A method of measuring local metabolism rate”, “A method for measuring water amount in the intercellular substance” and “A method for determining osmotic pressure of the intercellular substance in the microcirculation system” open principally new possibilities for early diagnosis of breast cancer. The method for early diagnosis breast cancer is also based on the method of diagnostics described in the section “A method for diagnosing a pathological state of the intercellular substance” and “A method for diagnosing a pathological state of internal organs”. These methods allow for performing diagnostics in the two possible practical modifications:

1) Additional diagnostics. In this variant the method is used as an additional method to the standard X-ray method;

2) Main diagnostics. In this variant the method is used as independent one on the other methods, individual method for diagnostics.

The method for early diagnosing breast cancer according to the variant “Additional diagnostics” supposes the following steps:

1) Detecting and localization of the tumor using the X-ray method;

2) measuring value of the parameter characterizing a state of the intercellular substance, for example, water amount in the intercellular substance, osmotic pressure or a resulting trans-capillary flow. Measurement is performed in two points (sites, zones) of the body surface—one immediately coinciding with the tumor projection region to the surface and another outside this region;

3) performing diagnostics by a differential drop value of the parameter in the two surface points.

The method for diagnostics may be also based on comparing values of the parameters obtained by measurements with the values thereof obtained by calculation. Such diagnostics stipulates the following additional steps:

4) measuring air temperature and blood sugar level;

5) determining a calculated value of the parameter characterizing a state of the intercellular substance;

6) determining a values' deviation of the parameters obtained my measurements from values of these parameters obtained by calculation by the values of air temperature and blood sugar level;

7) determining a character and degree of a pathological sate of the local site's intercellular substance by typical deviations of the parameters' values.

Physiological changes occurring in a tissue during the development of cancer tumor lead also to a change in the character of dynamic reactions of the intercellular substance in response to different physiological effects. In particular, a reaction of the intercellular substance to the effect of weak thermal flows and external pressures is modified. A local tissue reaction in response to a sugar load is also modified. These features open additional possibilities for diagnosing breast cancer. Such diagnostics is based on recording a time course of the parameter characterizing a state of the intercellular substance under the conditions of various physiological effect and it stipulates the following additional or independent steps:

5) a real-time measuring a value of the parameter characterizing a state of the intercellular substance (for example, water amount in the intercellular substance);

6) a local dosed effect on a tissue using physical factors of a weak intensity (examples of physical factors are an external thermal effect, external pressure, a direct electric current and a constant magnetic field, a sugar load);

7) a real-time measurement of a dynamic reaction of a recorded parameter in response to an external effect (for example, to a heat flow effect);

8) diagnosing a pathological state (intensity of reaction, time delay, time course character) by a character of the dynamic reaction.

The method for early diagnostics of breast cancer according to the variant “the main diagnostics”, unlike the variant “the additional diagnostics”, instead of the step 1) stipulates the following step:

1) a real-time recording a spatial-temporal distribution of the parameter characterizing a state of the intercellular substance. The methods of dymanic mapping are described in the section “A method for diagnosing a pathological state of internal organs”.

A real-time recording the parameter characterizing a state of the intercellular substance allows for localizing (at the first step) a region with modified tissue characteristics. Following a spatial localizing a problematic surface region, breast cancer is diagnosed using the subsequent steps described above.

Examples of a practical embodiment of the method are presented in FIGS. 27-29. The diagrams presented in FIG. 25, explain the principle of a real-time recording the parameters characterizing a state of two spatially separated local tissue sites. In the instant case, the recorded parameter is a tissue local metabolism rate (heat production).

Temporal changes in blood sugar level and as a sequence, tissue sugar consumption rate and heat production, were caused by performing a glucose tolerance test. The red and blue diagrams are the monitoring curves plotted using an experimental instrument manufactured in a variant of a two-channel micro calorimeter. The arrow marks the time moment of oral sugar load.

The distance between measuring sensors is 1.2 cm. Originating from the analysis of the curves, temporal changes in heat production of the two closely located tissue sites are seen to be practically synchronous. A temporal delay between the monitoring curves does not exceed 100 seconds.

The present experiment convincingly demonstrates that signal to noise ratio and accuracy of the micro calorimeter allow for detecting small differential differences of metabolism rate in two different but closely located tissue sites.

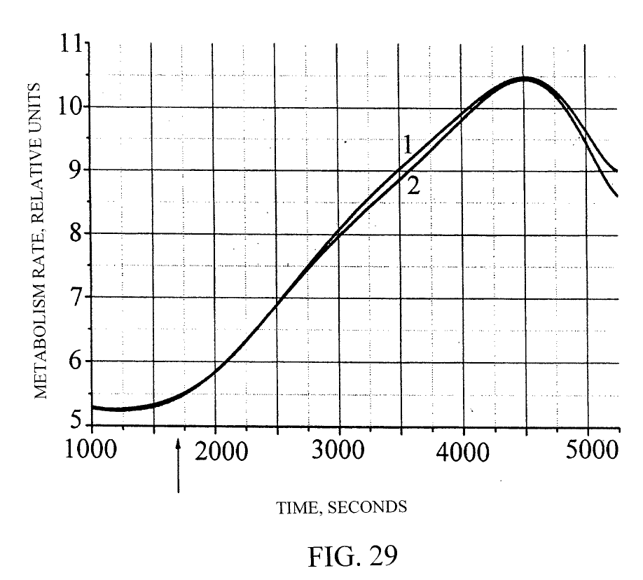

The measurement were performed using the experimental two-channel micro calorimeter the operation principle of which is described in the section “A method for measuring a local tissue metabolism”. The developed micro calorimeter allows for performing measurements of a tissue heat production with a high accuracy. The micro calorimeter allows for recording weak changes in heat production with sensitivity of 0.002 mcal/second.cm². FIGS. 28-29 present the experimental results explaining the principle of a dynamic mapping the parameter characterizing a state of the intercellular substance.

A high accuracy and a spatial detection provided by the micro calorimeter, allow for using it to detect malignant tumors and early medical diagnostics of breast cancer.

### A Method for Visualization of a Therapeutic Effect

The methods for measuring a tissue local metabolism rate and micro circulation parameters of a local tissue site open principally new possibilities for visualizing therapeutic effects as well as allow for a real-time determining efficacy of therapeutic procedures.

The method for visualization of a therapeutic effect stipulates the following steps:

A therapeutic effect is exerted in the regiment of a continuous monitoring the parameter characterizing a state of a local tissue site (microcirculation and metabolism rate) and a real-time recording the reaction of the controlled parameter is performed. Efficacy of a therapeutic effect is determined by typical characteristics of a time course of a recorded parameter (reaction or response to the effect). The described method is applicable for visualizing practically all kinds of therapeutic effects including the both drug effects and such effects as physiotherapeutic effects, the effect of acupuncture methods, homeopathy and others. The method is applicable for visualizing the both systemic effect on a whole body and local effects on different regions of the body tissues.

In particular, the instant method allows for visualizing the effects of the traditional physiotherapy which now includes such methods of physiotherapeutic effect as a local decompression, a constant magnetic field, electric current, ultrasound, electromagnetic radiation of optic and infrared range and others.

The described method provides for the possibility of not only visualizing a therapeutic effect but also optimizing regimens and doses of therapeutic effect in order to optimize a therapeutic effect in the real-time feedback regimen.

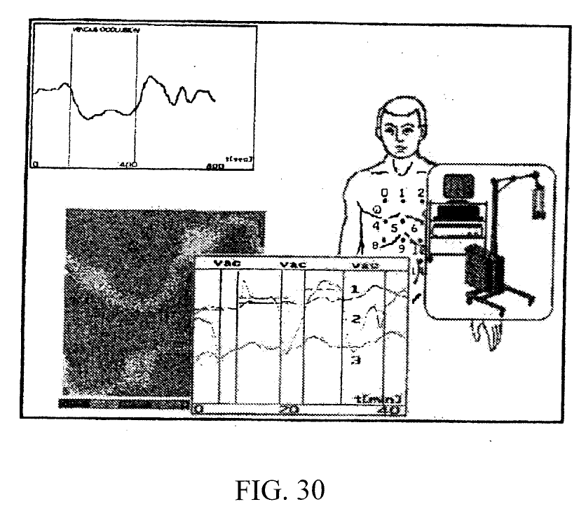

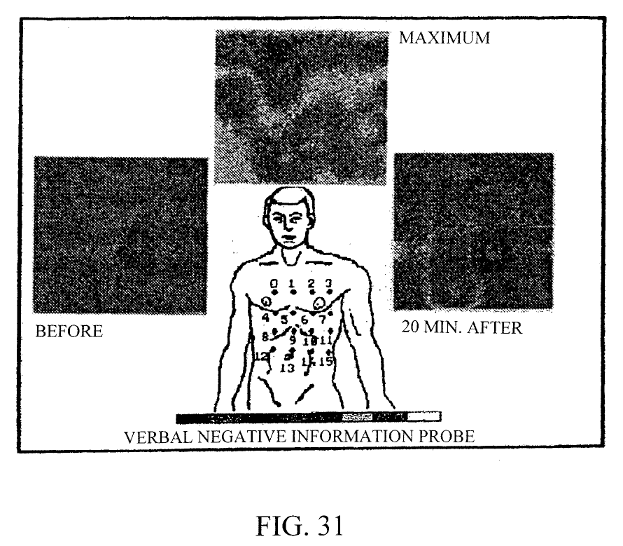

FIGS. 30-31 present the experimental results explaining the method for visualizing a therapeutic effect described above.

### Examples of Practical Use

The appearance of the experimental instrument the operation principle of which is described in the section “A method for measuring water amount in the intercellular substance using the electrometric method”, is shown in FIG. 7. The equivalent electric circuit explaining their measurement principle is shown in FIG. 6.

The developed technology allows for diminishing electronic components of the instrument down to the dimensions of one integral micro scheme and by this, to diminish dimensions of the instrument supposed for a practical use down to the dimensions not exceeding a wristwatch dimensions.

### Examples of Practical Use

### Results of Clinical Tests

Comparative measurements have been carried out on four patients: one practically healthy patient and three patients with diabetes (two patients with type 1 diabetes and one patient with type 2 diabetes).

The measurements were carried out using an experimental instrument in a continuous monitoring regimen (one measurement every 5-10 seconds) in duration of experiments of 30 to 150 minutes.

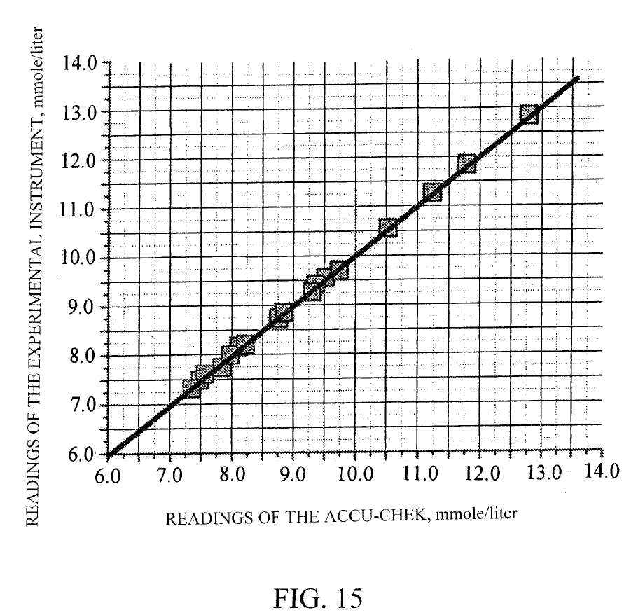

Calibration of the experimental is performed individually for every patient by four measurements performed by blood samples drawn form hand fingers. The number of control measurements by blood samples drawn from hand fingers during each experiment was from 2 to 9 measurements. The control measurements by blood samples drawn from a finger were carried out using the glucometer Accu-Chek Active (Roche Diagnostics GmbH, Roche Group). A total of 26 experiments were carried out with a total amount of control measurements making up 101. The results of the comparative experiments are presented in FIGS. 9-14 (“The study results on a practically healthy patient”) and FIGS. 15-20 (“The study results on patients with diabetes”).

### Examples of Practical Use

### The Study Results on a Practically Healthy Patient

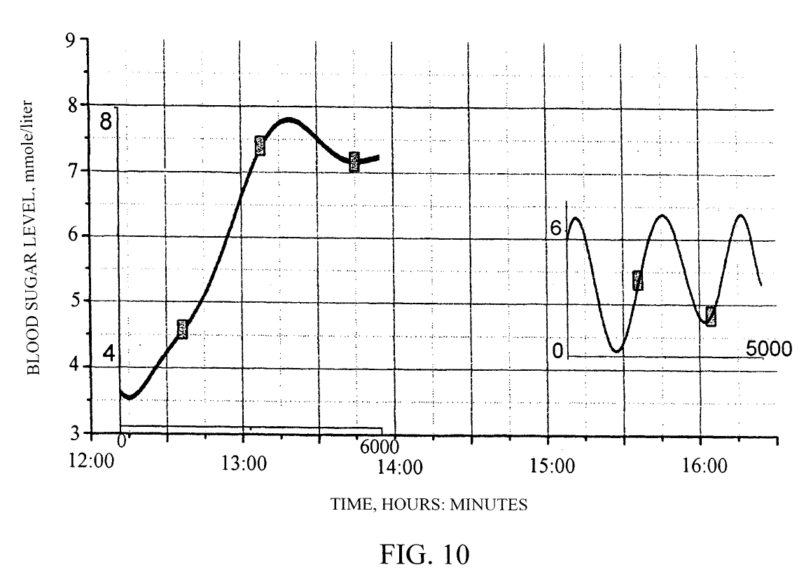

FIG. 9 presents the correlation diagram of the experimental instrument readings with the readings of the invasive glucometer by the results of 15 experiments conducted on one practically healthy patient. The control measurements were carried out using the glucometer “Accu-Chek Active”. A total amount of the control measurements by blood samples in 15 experiments was 38 measurements. All the measurements were done using one calibration. Readings of the experimental instrument at the time moments corresponding to the time moments of invasive by samples drawn from a finger, coincide with the readings of the certified glucometer with accuracy of 1-2% determined by error of the latter. Typical results of such experiments performed at different time during a day as well as at different days are presented in FIGS. 10-14.

FIG. 10 presents typical results of the comparative measurements: measuring a time course of blood sugar level, performed using the experimental instrument in the monitoring regimen (the red curve, rate of measurements 5-10 seconds) and the standard glucometer “Accu-Chek Active” manufactures by the firm Roche Diagnostics GmbH (the gray rectangles). Accuracy of the glucometer “Accu-Chek Active” measuring blood sugar level by photometric method (by blood samples drawn from a finger) is 1-2%. The diagrams present the results of two experiments on measuring blood sugar level in a practically healthy patient during a day: the first curve (from 12:00 to 13:30) is a change in blood sugar level caused by sugar load (the sugar curve); the second curve (from 15:10 to 16:15) is a time course of blood sugar level approximately 30-40 minutes after food intake during dinner. A total amount of measurements by blood samples in these experiments is 7 measurements (at the time moment 13:20 during the first experiment three measurements from one sample were performed).

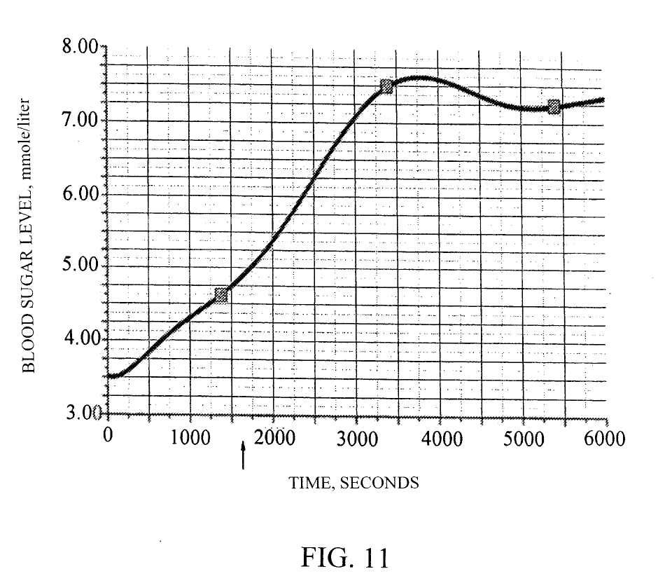

FIG. 11 presents a time course of blood sugar level caused by the standard sugar load (the glucose tolerance test or “The sugar curve”) (the first of two diagrams presented in FIG. 10). The red curve is the time course of blood sugar curve recorded in the monitoring regimen using the experimental instrument; the results of the control measurements performed using the “Accu-Chek Active” are shown by gray squares. The arrow marks the moment of the sugar load administration.

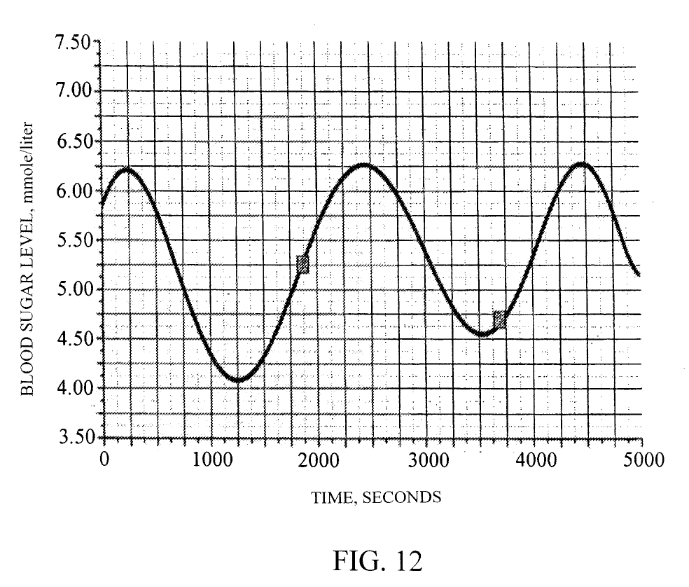

FIG. 12 presents the recording results of the time course of blood sugar level 30 minutes after dinner (the second of the two diagrams presented in FIG. 10).

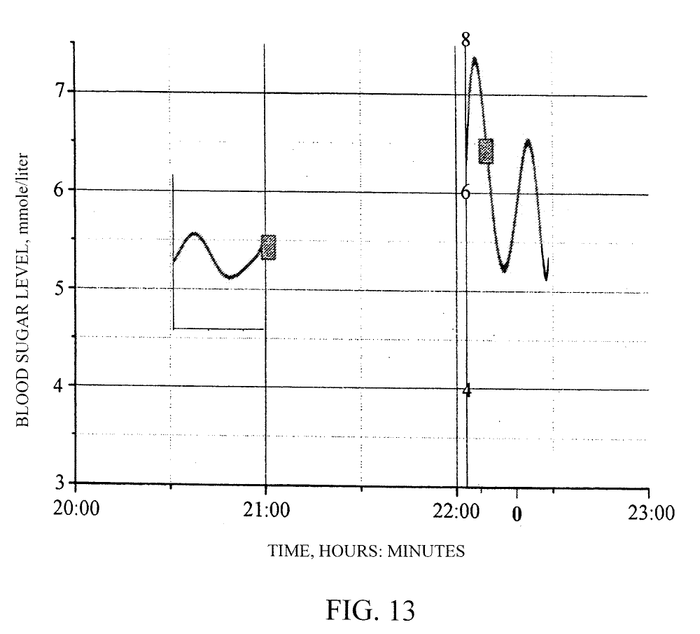

The diagrams of FIG. 13 present the results of two experiments (before and after supper) on blood sugar level measurement in the practically healthy patient: the first curve (from 20:30 till 21:00)—changes in blood sugar level prior to supper; the second curve (from 22:00 till 22:30) is the time course of blood sugar level approximately 20-30 minutes after supper.

FIG. 14 presents the recording results of blood sugar level time course during the standard glucose tolerance test procedure (“A sugar curve”). The arrow marks the moment of the sugar load administration.

### Examples of Practical Use

### The Results of Studies on Patients with Diabetes

The studies have been carried out in a clinical setting on three patients (males and females) with diabetes: two patients with type 1 diabetes and one patient with type 2 diabetes.

Measurements were carried out using the experimental instrument in the continuous monitoring regimen in duration of the experiments from 30 to 60 minutes. Control measurements by blood samples drawn from hand fingers during each experiment amounted from 4 to 9 measurements.

Control measurements by blood samples drawn from hand fingers were carried out using the glucometer Accu-Chek Active (Roche Diagnostics GmbH, Roche Group). A total of 11 experiments with a total amount of 63 control measurements were carried out. The typical experimental results are presented in FIGS. 15-20.

### Examples of Practical Use

### The Results of Pilot Studies on Patients with Diabetes. A Patient (D!) with Type 1 Diabetes

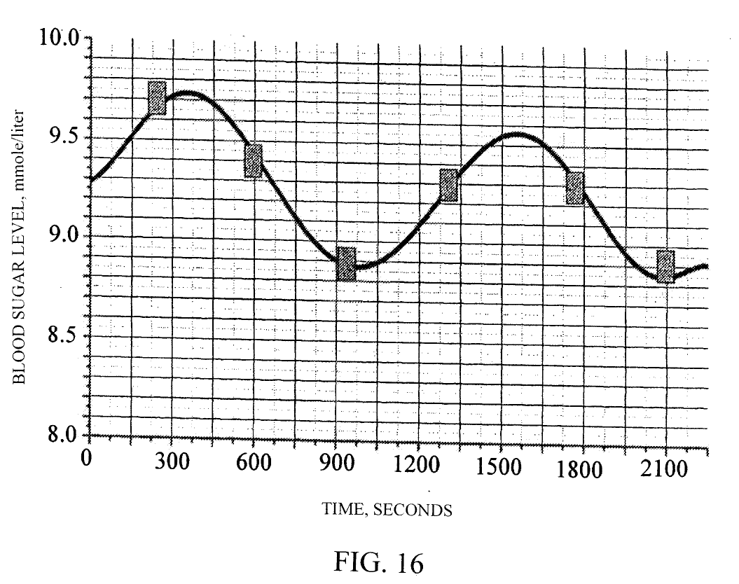

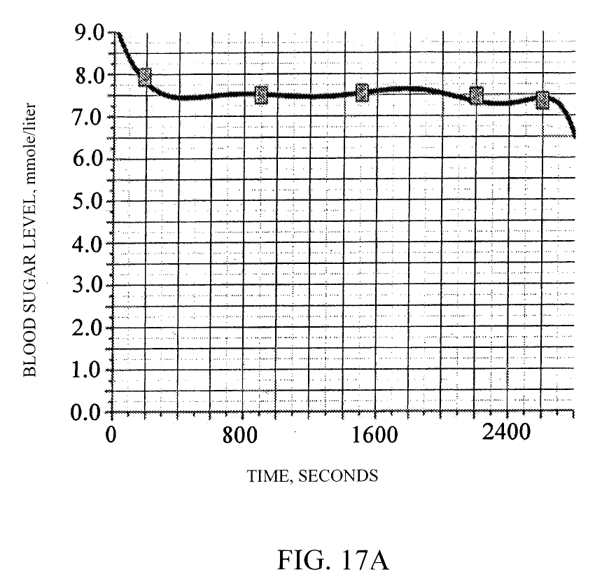

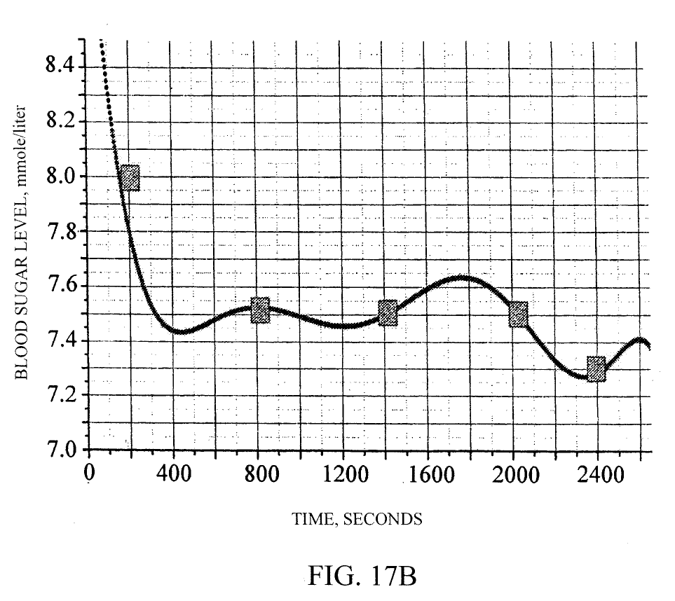

FIG. 15 presents the correlation diagram between readings of the experimental instrument by the result of four experiments carried out on one patient D1 with type 1 diabetes (a 55 year old woman). Control measurements were carried out using the glucometer “Accu-Chek Active”. Control measurements by blood samples in four experiments total 21 measurements. All the measurements were carried out with one calibration. Readings of the experimental instrument at the time moments corresponding to the time moment of a control measurement by blood samples drawn from a finger coincide with readings of the certified glucometer with accuracy determined by an error of the latter (1-2%). Typical results of these experiments carried out at different days are presented in FIGS. 16-17.

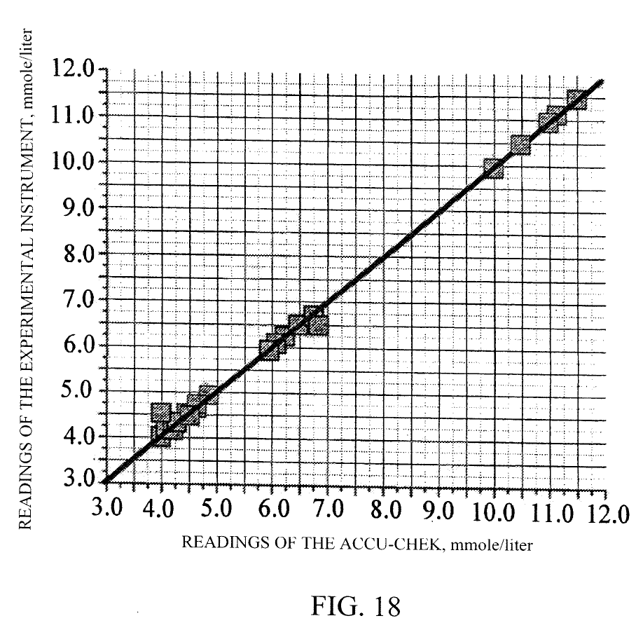

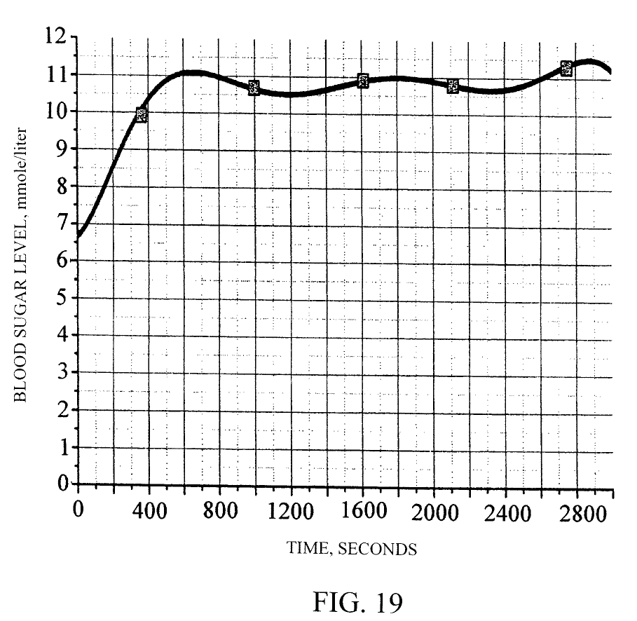

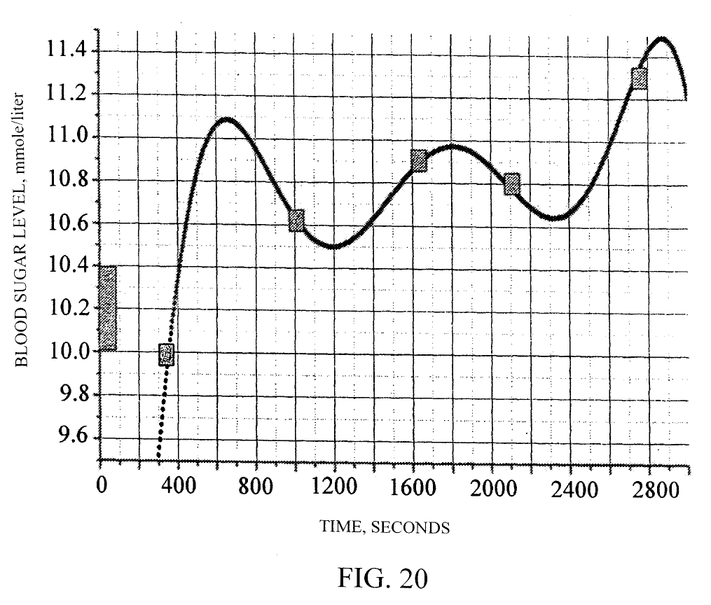

A patient with type 2 diabetes. FIG. 18 presents the correlation diagram of the experimental instrument's readings with readings of the invasive glucometer by the results of four experiments carried out on one patient with type 2 diabetes (a 76 year old man). Control measurements were carried out using the glucometer “Accu-Chek Active”. Control measurements by blood samples in four experiments total 21 measurements. All the measurements were carried out with one calibration. Readings of the experimental instrument at the time moments corresponding to the time moment of a control measurement by blood samples drawn from a finger, coincide with readings of the certified glucometer with accuracy determined by an error of the latter (1-2%). Typical results of these experiments carried out at different days are presented in FIGS. 19-20.

### BIBLIOGRAPHY

1. Edsall J. T., Cutfreind H. BIOTHERMODYNAMICS. The Study of Biochemical Processes at Equilibrium. JOHN WILLEY & SONS, 1983.
2. Kendysh I. N. Regulyatsiya uglevodnogo obmena. Izdatel'stvo “Meditsina”, Moskva (In Russian).
3. Peisner Leonardo. Concepts in Bioenergetics. Peusner Biomedical Associates, Prentice—Hall, Inc., Englewood Cliffs, N.J., 1974.
4. Lehninger A. L. BIOCHEMISTRY. The John Hopkins University, Schoo; of Medicine. Worth Publishers, Inc., New York, 1972.
5. Atkins P. W. The second law. Scientific American Library, New York, 1984.
6. Musin R. F. PATENT RD No 2087128 na izobreteniye “Ustrojstvo dlya izmereniya electricheskikh svojstv rogovogo sloya epidermisa”. (In Russian).
7. Musin R. F., Morozov V. A., Gogik E. E., Gulyaev Yu. V. (1986). Electricheskiye svojstva rogovogo sloya epidermisa cheloveka I transport vody v nyom. —Biophyzika, t. 31, vyp.3, s. 478-480. (In Russian).
8. Forbath N., Hetenyi C. Glucose dynamics in normal subjects and diabetic patients before and after a glucose load. —Dfiabetes, 1966, vol. 15, #11, p. 778-789.
9. Hall S. E. H., Saunders J., Sonksen P. H. Glucose and free acid turnover in normal subjects and diabetic patients before and after insulin treatment. —Diabetologia, 1979, vol. 16, #5, p. 297-306.
10. Musin R. F., Godik E. E., Gulyaev Y. V., Moroziov V. A. Natural water diffusion through the stratum corneum of the human body epidermis and its electrical properties/—3 International conference on water and ions in biological systems. Bucharest, Romania, 1984, p. 34.
11. Musin R. F., Ivanova N. Yu., Martynov V. A., Morozov V. A., Godik E. E., Gulyaev Yu. V. (1986). 0 chuvstvitel'nosti kozhi chelioveka k infrakrasnym teplovym potokam. —Doklady akademii nauk SSSR, tom 289, No 3, s.718-720. (In Russian).
12. Gulyaev Yu. V., Godik E. E., Musin R. F., Morozov V. A., Martynov V. A., Valiev I. V. (1989). Porogi tep[lovoy chuvstvitel'nosty kozhi k electromagnitnym izluchenijam. —Sensornie systemy, tom 3, No 2, s.209-212. (In Russian).
13. Godik E. E., Morozov V. A., Musin R. F. (1985). Odinamike relaksatsii tribozaryada na poverkhnosti rogovogo sloya epidermisa kozhi. —Biophysika, tom 30, vyp.2, s.309-312. (In Russian).
14. Musin R. F., Godik E. E., Gulyaev Yu. V., Morozov V. A., Sudarev A. M. Membrane mechanisms of water transport in epidermis. 4th International conference on water and ions in biological systems. Bucharest, Romania, 1988, pp. 167-172.
15. Musin R. F., Morozov V. A., Sudarev A. M. (1990). O mekhanizmakh transporta vody v epidermise. —Biophyzika, tom 35, vyp.4, s.653-656. (IN Russian).
16. Yas Kuno. (1959). Human Perspiration. Charles & Thomas Publ., Springfield, Ill., USA.
17. A. V. Korobkov, S. A. Chesnokova. (1986). Atlas Po normal'noy physiologii. M.: Vysshaya shkola, 351 s. (In Russian)
18. Gomeostaz. Pod red. P. D. Gorizontova. M., “Meditasina”, 1976, 464 s., il. (In Russian).
19. U.S. Pat. No. 5,795,305, Aug. 18, 1998, Cho et al. Process and device for non-invasive determination of glucose concentration in parts of human body.
20. U.S. Pat. No. 5,823,966, October 1998, Buchet. Non-invesive continuous blood glucose monitoring.
21. U.S. Pat. No. 5,890,489, Apr. 6, 1999, Elden. Method for non-invasive determination of glucose in body fluids.
22. U.S. Pat. No. 5,924,996, Jul. 20, 1999, Cho et al. Process and device for detecting the exchange of heat between the human body and the invented device and its correlation to the glucose concentration in human body.
23. U.S. Pat. No. 6,522,903, Feb. 18, 2003, Berman et al. Glucose measurement assessment utilizing non-invasive assessment methods.
24. U.S. Pat. No. 6,517,482, Feb. 11, 2003, Elden et al. Method and apparatus for non-invasive determination of glucose in body fluids.
25. U.S. Pat. No. 5,040,541, Aug. 20, 1991, Poppendiek. Whole body calorimeter.
26. U.S. Pat. No. 4,386,604, Jun. 7, 1993, Herchey. Determination of the basal metabolic rate of humans with a whole body calorimeter.

1-277. (canceled)

278. A device for the measurement of a tissue metabolism intensity on a local site characterized in that the device is equipped with a sensor for measuring vapor flow density of water evaporating from a limited skin site surface in the process of a non-perceivable perspiration with a heat flow sensor and a measuring unit comprising devices for processing and displaying signals from the sensors.

279. The device according to claim 278 characterized in that it is additionally equipped with a measuring capsule with a sensor for measuring a total amount of water evaporating from a skin surface in the process of a non-perceivable perspiration, and with a temperature sensor.

280. The device according to claim 279 characterized in that it comprises a device for generating a dosed pressure on a skin surface and the measuring capsule comprises a sealed cavity a working surface of which contacting with the skin is manufactured in the form of a rigid membrane which is permeable or semi-permeable for water.

281. The device according to claim 279 characterized in that the measuring capsule comprises a cavity having a diffusion contact with a skin surface and having no mechanical contact with the skin surface.

282. The device according to claim 279 characterized in that it is equipped with a water-impermeable applicator applied to the skin corneous layer surface using an appliance for generating a dosed pressure, and a sensor for measuring water amount in a tissue volume located under the applicator.

283. The device according to claim 282 characterized in that the sensor for measuring water amount is a sensor for measuring water amount in the skin epidermal corneous layer.

284. The device according to claim 283 characterized in that the sensor for measuring water amount in the corneous layer is an electrometric sensor measuring electrical characteristics of the corneous layer.

285. The device according to claim 284 characterized in that it additionally comprises a basic and a measuring electrodes, an appliance for generating a dosed pressure of the electrodes on the skin surface, a power supply and a measuring unit, and at least one of the electrodes is manufactured in the form of a dry water-impermeable electrode.

286. The device according to claim 282 characterized in that the sensor for measuring water amount in a tissue volume under the applicator is based on measuring a tissue parameter selected form the group including a tissue pressure, a hydraulic pressure in the microcirculation system, an elastic pressure, a temperature and spectral characteristics.

287. The device according to claim 278 characterized in that it additionally comprises a sensor selected from the group consisting of an atmospheric pressure sensor, an excessive pressure sensor, an air humidity sensor, a skin surface temperature sensor and combinations thereof.

288. The device according to claim 280 or 285 characterized in that the appliance for generating a dosed pressure is manufactured using the pneumatic, mechanical, piezoelectric, electromagnetic, vacuum or hydraulic principle or combinations thereof.

289. The device according to claim 278 characterized in that it comprises a source of a calibrated thermal power.

290. The device according to claim 289 characterized in that the source of a calibrated thermal power is manufactures in the form of a device using an element which is selected from the group consisting of an electric resistance, an element operating on the basis of the Peltier's effect and a photodiode.

291. The device according to claim 278 characterized in that it is designed for measuring a tissue parameter selected from the group consisting of blood glucose level, elastic pressure of the intercellular substance, water amount in the intercellular substance, capillary pressure, tissue pressure, osmotic pressure of the intercellular substance, a resulting transcapillary pressure, blood pressure or a combination of said parameters.

292. The device according to claim 291 characterized in that it additionally comprises an appliance for a dosed effect on a tissue site using physical factors.

293. The device according to claim 283 characterized in that it additionally comprises a sensor for measuring glucose concentration in the epidermal corneous layer.

294. The device according to claim 283 characterized in that the device is equipped with a water-impermeable applicator applied to a surface of the skin corneous layer using an appliance for generating a dosed pressure, and a temperature sensor and a sensor for measuring concentration of a blood biochemical component in the epidermal corneous layer.

295. The device according to claim 292 characterized in that it additionally comprises an appliance for exerting a local dosed physical effect on a tissue site and a sensor of a parameter characterizing the tissue site state.

296. The device according to claim 295 characterized in that it additionally comprises a feedback sensor for controlling a state of a tissue site subjected to effect.

297. The device according to claim 278 characterized in that it additionally comprises an air temperature sensor as a heat flow sensor, and a sensor of a parameter characterizing the cardiovascular system.

298. The device according to claim 297 characterized in that it additionally comprises a sensor of a parameter characterizing the cardiovascular system selected from the group including sensors of heart rate, cardiac output, blood flow velocity in a tissue site subjected to effect, blood pressure, pressure in the microcirculation system, capillary pressure, a resulting trans-capillary flow, tissue or osmotic pressure of the intercellular substance, elastic pressure of the intercellular substance or elastic strain, a hydraulic resistance of capillary vessels, water amount in the intercellular substance or a combination of said sensor.

299. The device according to claim 291 or 297 characterized in that it additionally comprises an appliance for exerting a local physical effect on a tissue site.

300. The device according to claim 299 characterized in that it additionally comprises an appliance for exerting a dosed thermal effect, an external pressure, a local decompression, electric current or a magnetic field or for a combination of said effects.

301. The device according to claim 297 characterized in that it additionally comprises an air temperature sensor, a blood glucose level sensor and a sensor for measuring of at least one of parameters characterizing the cardiovascular system.

302. The device according to claim 301 characterized in that it additionally comprises a sensor selected from the group including sensors of heart rate, cardiac output, blood pressure, pressure in the microcirculation system, a resulting trans-capillary flow, water amount in the intercellular substance or osmotic pressure of the intercellular substance or a combination of said sensors.
